(19) United States
(12) Patent Application Publication
(10) Pub. No.: US 2021/0219853 A1
(43) Pub. Date: Jul. 22, 2021

(54) BELT AND BLOOD PRESSURE MEASUREMENT DEVICE

(71) Applicants: OMRON HEALTHCARE Co., Ltd., Kyoto (JP); OMRON Corporation, Kyoto (JP)

(72) Inventors: Noboru KOHARA, Kyoto (JP); Tomoyuki NISHIDA, Kyoto (JP); Hirokazu TANAKA, Kyoto (JP); Shinji MIZUNO, Kyoto (JP); Kotaro KITAJO, Kyoto (JP); Takashi ONO, Kyoto (JP)

(21) Appl. No.: 17/223,540

(22) Filed: Apr. 6, 2021

Related U.S. Application Data
(63) Continuation of application No. PCT/JP2019/038341, filed on Sep. 27, 2019.

(30) Foreign Application Priority Data
Oct. 23, 2018 (JP) 2018-199476

(51) Int. Cl. A61B 5/021

(52) U.S. Cl. CPC A61B 5/02141; A61B 2560/04

(57) ABSTRACT

A belt providing improved durability and a blood pressure measurement device. A second belt of a belt of a blood pressure measurement device includes a second belt body constituted in a band-like shape using a resin material and including a plurality of small holes formed along a longitudinal direction, and a second insert being disposed in the second belt body and including a plurality of holes with a plurality of the small holes disposed on an inner side of the respective holes, a cross section of a portion of the second insert, which is between two adjacent holes and orthogonal to a width direction of the second belt body, being constituted like a trapezoid with a short side being disposed on a living body side, and the second insert being formed of a material having a higher tensile strength than the resin material constituting the second belt body.

### CROSS-REFERENCE TO RELATED APPLICATIONS

This application is the U.S. national stage application filed pursuant to 35 U.S.C. 365(c) and 120 as a continuation of International Patent Application No. PCT/JP2019/038341, filed Sep. 27, 2019, which application claims priority from Japanese Patent Application No. 2018-199476, filed Oct. 23, 2018, which applications are incorporated herein by reference in their entireties.

### TECHNICAL FIELD

The present invention relates to a belt and a blood pressure measurement device for measuring blood pressure.

### BACKGROUND ART

In recent years, blood pressure measurement devices for measuring blood pressure are being used to monitor health status at home, as well as in medical facilities. A blood pressure measurement device detects vibration of the artery wall to measure blood pressure by, for example, inflating and contracting a cuff wrapped around the upper arm or the wrist of a living body and detecting the pressure of the cuff using a pressure sensor.

Additionally, a technique is known in which a belt is prevented from being elongated when the cuff is inflated by providing, in the belt, an insert formed of a material having a higher tensile strength than the belt.

### CITATION LIST

### Patent Literature

Patent Document 1: JP 2017-121479 A

### SUMMARY OF INVENTION

### Technical Problem

When the insert is formed from a plate-like member, for example, by stamping using pressing, burrs may be formed at edges of the insert. In a case where the insert includes burrs at the edges, when stress is applied to the belt during use in the blood pressure measurement device, the stress may concentrate at the burrs and cracking may occur in the vicinity of the burrs in the belt.

Thus, an object of the present invention is to provide a belt that can provide improved durability and a blood pressure measurement device.

### Solution to Problem

According to one aspect, a belt is provided that includes a first belt constituted in a band-like shape, a second belt including a belt body and an insert, the belt body being constituted in a band-like shape using a resin material and including a plurality of first holes formed along a longitudinal direction, the insert being disposed in the belt body and including a plurality of second holes with a plurality of the first holes disposed on an inner side of the respective second holes, a cross section of a portion of the insert, which is between two adjacent second holes of the plurality of second holes and orthogonal to a width direction of the belt body, being constituted like a trapezoid with a short side being disposed on a living body side, the insert being formed of a material having a higher tensile strength than the resin material, and a connector connecting the first belt and the second belt.

Here, the tensile strength indicates the degree of elongation with respect to a tensile load. A material with high tensile strength is, as an example, a resin material, and examples of the resin material include high strength polyarylate fibers, liquid crystal polymers, PET resins, and PEN resins. Additionally, here, the living body is, for example, the wrist or the upper arm.

According to this aspect, a cross-section of the portion of the insert between the second holes is constituted like a trapezoid, and a main surface of the portion between the second holes including a short side of the trapezoid is disposed on a living body side with respect to a main surface of a portion of the insert between the second holes including a long side of the trapezoid, and inclined side surfaces connecting the both main surfaces face the first holes in the belt body.

Thus, in a state in which the belt is attached to the living body, when a load is applied from the living body side of the second hole, the portion of the insert between the second holes receives a load on the side surfaces. This enables suppression of concentration of stress around the portion of the insert between the second holes, thus allowing suppression of cracking of the belt body As a result, durability of the belt can be improved.

According to the one aspect described above, the belt is provided in which, in the blood pressure measurement device, the insert includes a positioning portion configured for positioning in a mold used to form the second belt.

According to this aspect, the insert can be positioned within the mold when the second belt is manufactured.

According to one aspect, a blood pressure measurement device is provided that includes a belt including a first belt constituted in a band-like shape, a second belt including a belt body and an insert, the belt body being constituted in a band-like shape using a resin material and including a plurality of first holes formed along a longitudinal direction, the insert being disposed in the belt body and including a plurality of second holes with a plurality of the first holes disposed on an inner side of the respective second holes, a cross section of a portion of the insert, which is between two adjacent second holes of the plurality of second holes and orthogonal to a width direction of the belt body, being constituted like a trapezoid with a short side being disposed on a living body side, the insert being formed of a material having a higher tensile strength than the resin material, and a connector connecting the first belt and the second belt, and a cuff structure provided on an inner side of the belt and configured to be inflated with a fluid.

According to this aspect, a cross-section of the portion of the insert between the second holes is constituted like a trapezoid, and a main surface of the portion between the second holes including a short side of the trapezoid is disposed on a living body side with respect to a main surface of a portion of the insert between the second holes including a long side of the trapezoid, and inclined side surfaces connecting the both main surfaces face the first holes in the belt body.

Thus, in a state in which the belt is attached to the living body, when a load is applied from the living body side of the second hole, the portion of the insert between the second holes receives a load on the side surfaces. This enables suppression of concentration of stress around the portion of the insert between the second holes, thus allowing suppression of cracking of the belt body. As a result, durability of the belt can be improved.

### Advantageous Effects of Invention

The present invention can provide a belt that can provide improved durability and a blood pressure measurement device.

### BRIEF DESCRIPTION OF DRAWINGS

FIG. 1 is a perspective view illustrating a configuration of a blood pressure measurement device according to a first embodiment of the present invention.

FIG. 2 is a perspective view illustrating the configuration of the blood pressure measurement device.

FIG. 3 is an exploded perspective view illustrating the configuration of the blood pressure measurement device.

FIG. 4 is an explanatory diagram illustrating a state in which the blood pressure measurement device is attached to the wrist.

FIG. 5 is a block diagram illustrating the configuration of the blood pressure measurement device.

FIG. 6 is a perspective view illustrating a configuration of a device body and a curler of the blood pressure measurement device.

FIG. 7 is a cross-sectional view illustrating a configuration of a first belt of the blood pressure measurement device.

FIG. 8 is a perspective view illustrating a configuration of a second belt of the blood pressure measurement device.

FIG. 9 is a partially cutaway perspective view illustrating the configuration of the second belt.

FIG. 10 is a plan view illustrating a second insert of the second belt.

FIG. 11 is a cross-sectional view illustrating a configuration of a buckle of the first belt.

FIG. 12 is an explanatory diagram illustrating an example of a method for manufacturing the second belt.

FIG. 13 is a plan view illustrating a configuration of a cuff structure of the blood pressure measurement device.

FIG. 14 is a plan view illustrating another configuration of the cuff structure of the blood pressure measurement device.

FIG. 15 is a cross-sectional view illustrating a configuration of the belt, the curler, and the cuff structure of the blood pressure measurement device.

FIG. 16 is a cross-sectional view illustrating the configuration of the curler and the cuff structure of the blood pressure measurement device.

FIG. 17 is a cross-sectional view illustrating the configuration of the curler and the cuff structure of the blood pressure measurement device.

FIG. 18 is an explanatory diagram illustrating the configuration in which the cuff structure is inflated in a state in which the blood pressure measurement device is attached to the wrist.

FIG. 19 is a cross-sectional view illustrating the configuration in which the cuff structure is inflated in a state in which the blood pressure measurement device is attached to the wrist.

FIG. 20 is a flowchart illustrating an example of usage of the blood pressure measurement device.

FIG. 21 is a perspective view illustrating an example in which the blood pressure measurement device is attached to the wrist.

FIG. 22 is a perspective view illustrating an example in which the blood pressure measurement device is attached to the wrist.

FIG. 23 is a perspective view illustrating an example in which the blood pressure measurement device is attached to the wrist.

FIG. 24 is a cross-sectional view illustrating a configuration of the vicinity of the buckle when the cuff structure is inflated in a state in which the blood pressure measurement device is attached to the wrist.

FIG. 25 is a cross-sectional view illustrating a configuration of a second belt of a blood pressure measurement device according to a second embodiment of the present invention.

FIG. 26 is a plan view illustrating a configuration of a second insert of a second belt of a blood pressure measurement device according to a third embodiment of the present invention.

FIG. 27 is a cross-sectional view illustrating a modified example of a configuration of a hole reinforcing portion of the second belt of the blood pressure measurement device according to the first to third embodiments of the present invention.

FIG. 28 is a cross-sectional view illustrating a modified example of the configuration of the hole reinforcing portion of the second belt of the blood pressure measurement device according to the first to third embodiments of the present invention.

FIG. 29 is a cross-sectional view illustrating a modified example of the configuration of the hole reinforcing portion of the second belt of the blood pressure measurement device according to the first to third embodiments of the present invention.

FIG. 30 is a cross-sectional view illustrating a modified example of the configuration of the hole reinforcing portion of the second belt of the blood pressure measurement device according to the first to third embodiments of the present invention.

FIG. 31 is a cross-sectional view illustrating a modified example of the configuration of the hole reinforcing portion of the second belt of the blood pressure measurement device according to the first to third embodiments of the present invention.

FIG. 32 is a cross-sectional view illustrating a modified example of the configuration of the hole reinforcing portion of the second belt of the blood pressure measurement device according to the first to third embodiments of the present invention.

### DESCRIPTION OF EMBODIMENTS

### First Embodiment

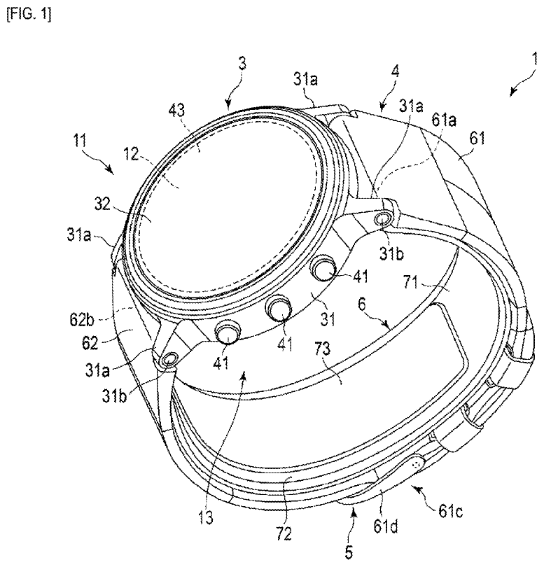

An example of a blood pressure measurement device 1 according to the first embodiment of the present invention is described below using FIGS. 1 to 16.

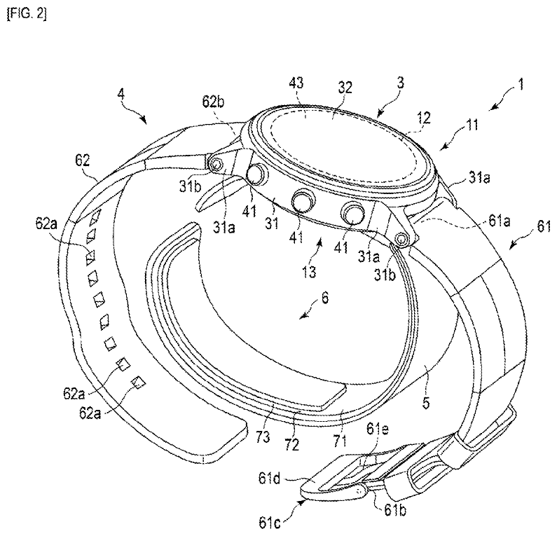

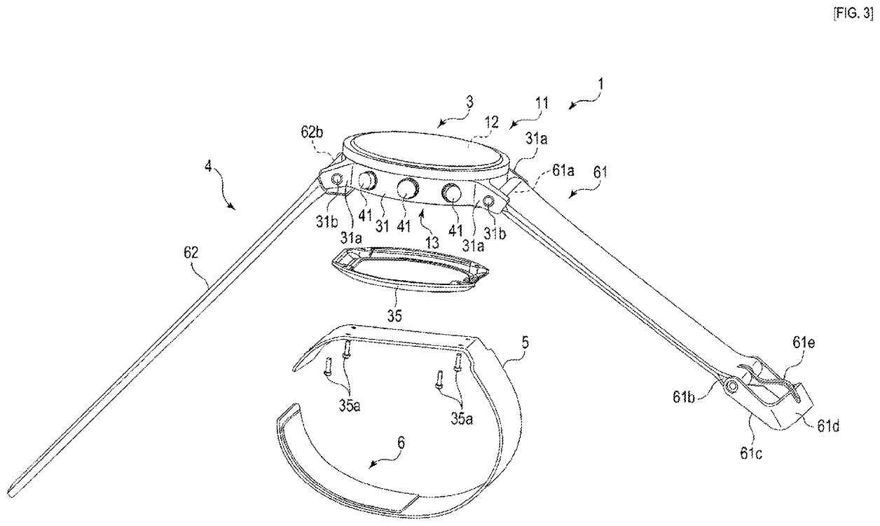

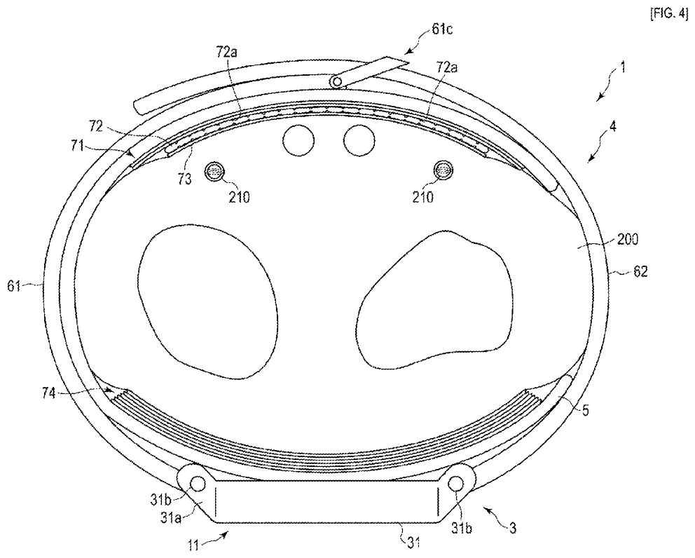

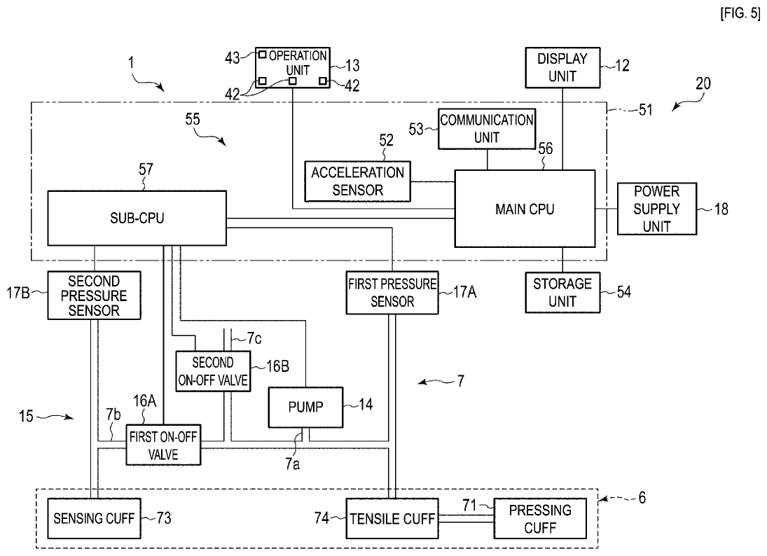

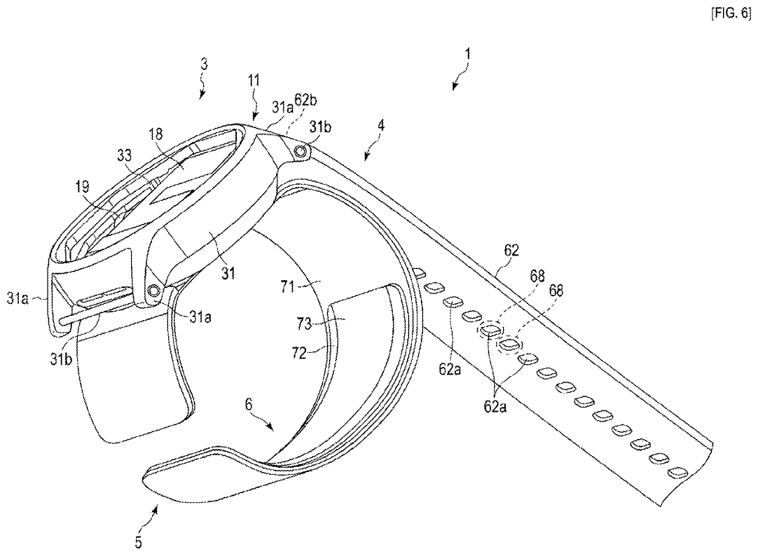

FIG. 1 is a perspective view illustrating a configuration of the blood pressure measurement device 1 according to an embodiment of the present invention in a state in which a belt 4 is closed. FIG. 2 is a perspective view illustrating the configuration of the blood pressure measurement device 1 in a state in which the belt 4 is open. FIG. 3 is an exploded perspective view illustrating the configuration of the blood pressure measurement device 1. FIG. 4 is an explanatory diagram illustrating, in cross section, a state in which the blood pressure measurement device 1 is attached to the wrist 200. FIG. 5 is a block diagram illustrating the configuration of the blood pressure measurement device 1. FIG. 6 is a perspective view illustrating a configuration of a device body 3 and a curler 5 of the blood pressure measurement device 1.

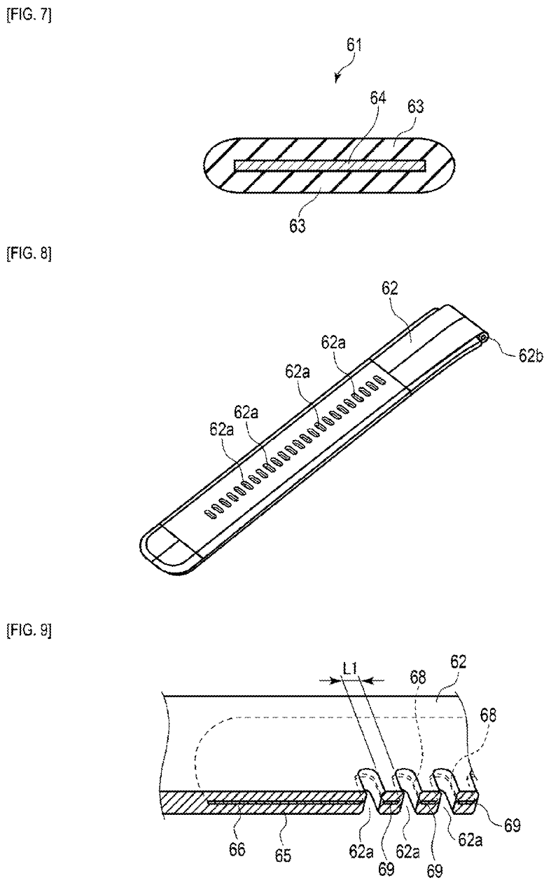

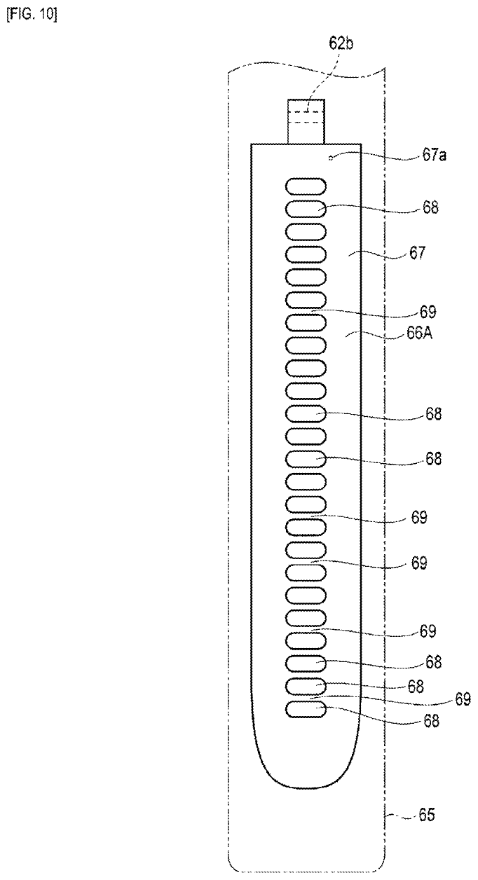

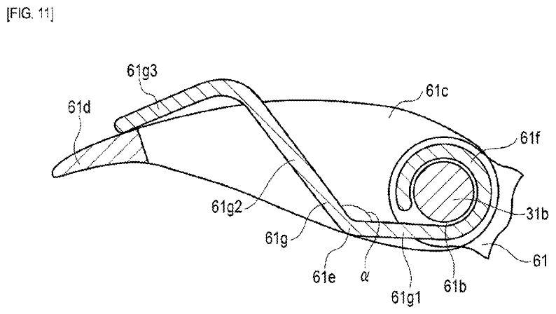

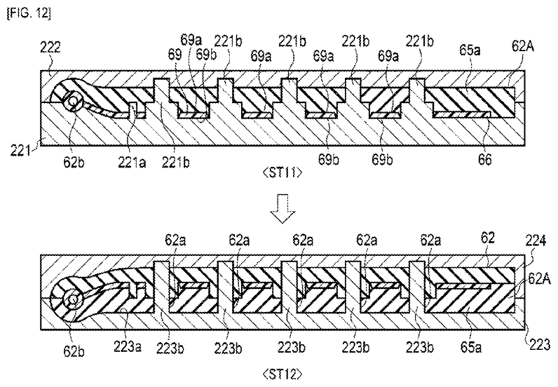

FIG. 7 is a cross-sectional view illustrating a configuration of a first belt 61 of the blood pressure measurement device 1 in a cross section orthogonal to the longitudinal direction of the first belt 61. FIG. 8 is a perspective view illustrating a configuration of a second belt 62 of the blood pressure measurement device 1. FIG. 9 is a partially cutaway perspective view illustrating the configuration of the second belt 62. FIG. 10 is a plan view illustrating a second insert 66 of the second belt 62. Note that in FIG. 10, a part of an outer shell of the second belt 62 is illustrated by a long dashed double-short dashed line. FIG. 11 is a cross-sectional view illustrating a configuration of a buckle 61c of the first belt 61. FIG. 12 is an explanatory diagram illustrating an example of a method for manufacturing the second belt 62.

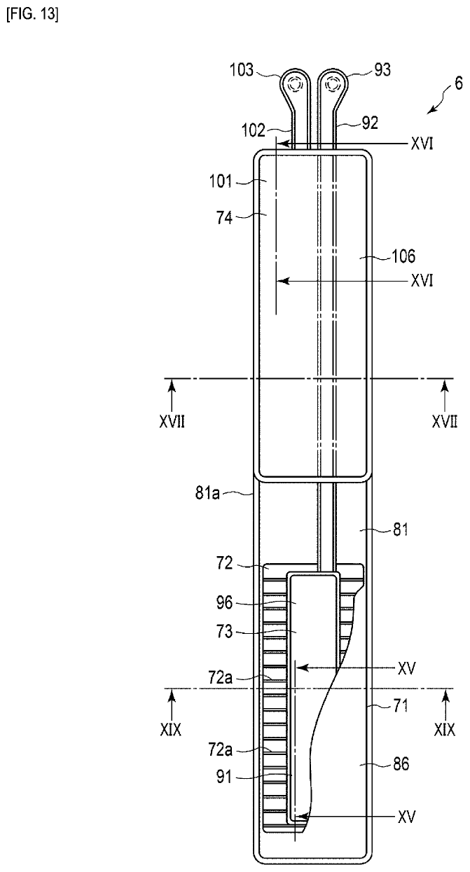

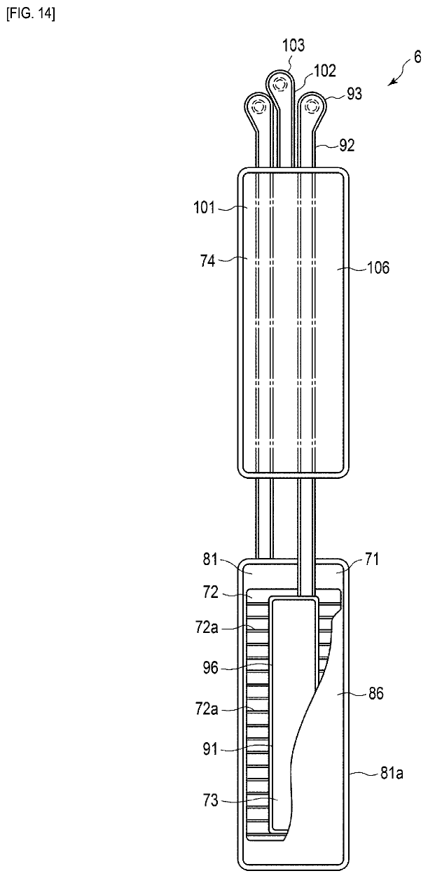

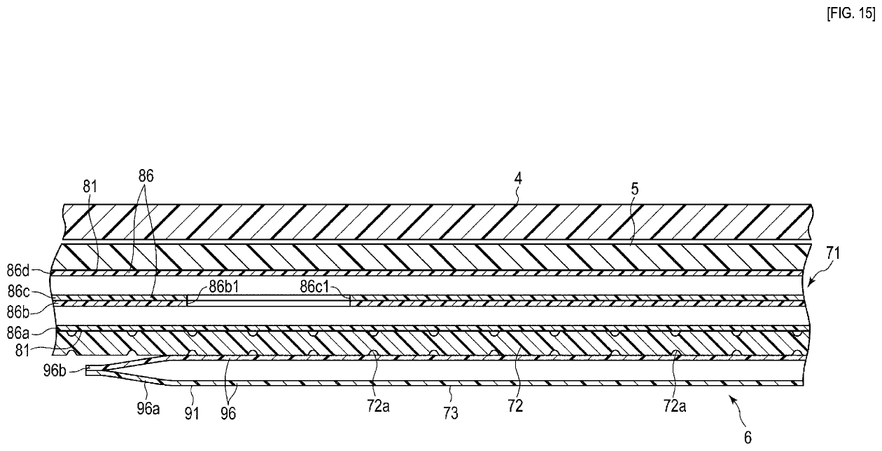

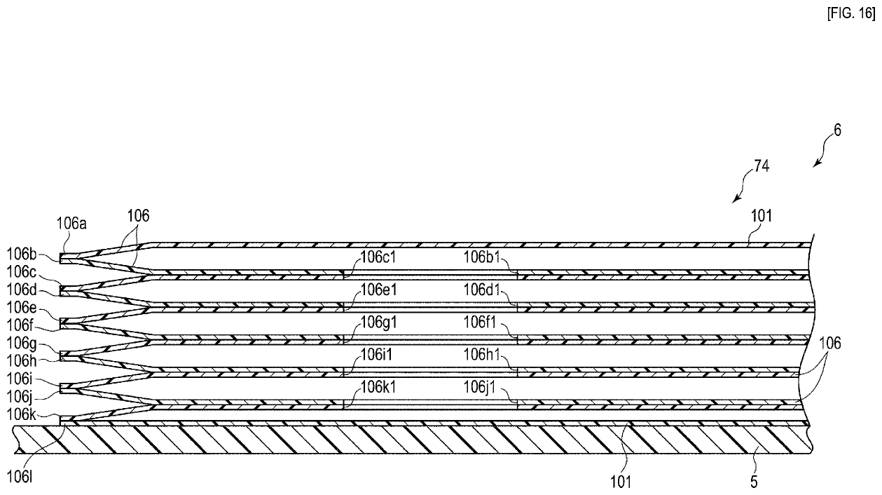

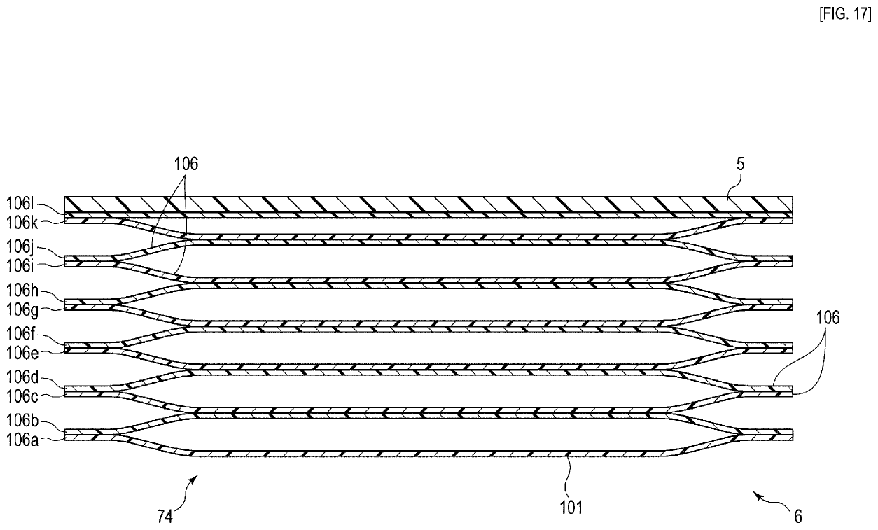

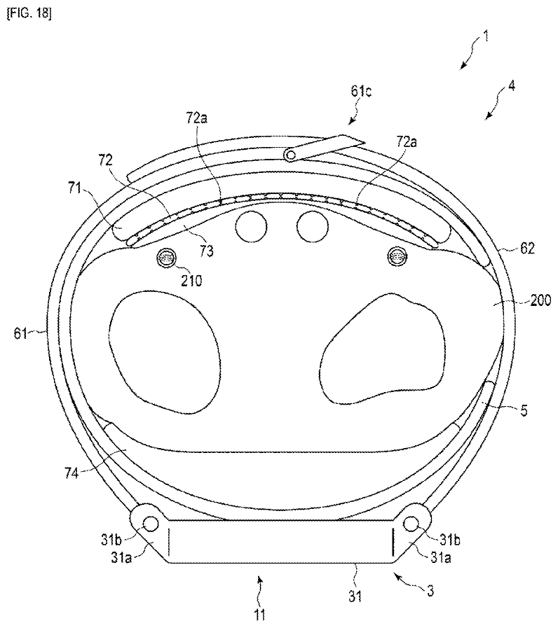

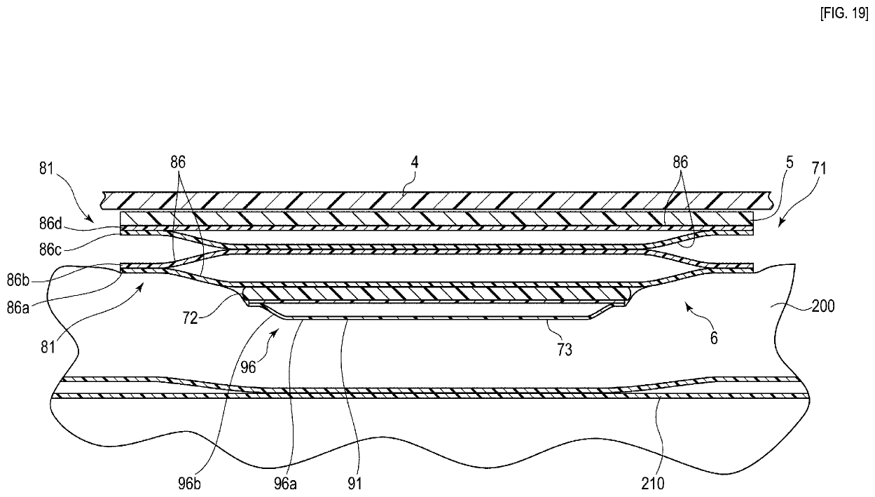

FIG. 13 is a plan view illustrating a configuration of a cuff structure 6 of the blood pressure measurement device 1. FIG. 14 is a plan view illustrating another configuration of the cuff structure 6 of the blood pressure measurement device 1. FIG. 15 is a cross-sectional view illustrating a configuration of the belt 4, the curler 5, and the cuff structure 6 on a palm-side cuff 71 side of the blood pressure measurement device 1 which is taken along line XV-XV in FIG. 13. FIG. 16 is a cross-sectional view illustrating a configuration of the curler 5 and the cuff structure 6 on a back-side cuff 74 side of the blood pressure measurement device 1 which is taken along line XVI-XVI in FIG. 13. FIG. 17 is a cross-sectional view illustrating a configuration of the cuff structure 6 with the curler 5 and a tube 92 omitted, on the back-side cuff 74 side of the blood pressure measurement device 1, which is taken along line XVII-XVII in FIG. 13. FIG. 18 is an explanatory diagram illustrating the configuration in which the cuff structure 6 is inflated in a state in which the blood pressure measurement device 1 is attached to the wrist 200. FIG. 19 is an explanatory diagram illustrating the configuration in which the cuff structure 6 is inflated in a state in which the blood pressure measurement device 1 is attached to the wrist, which is taken along line XIX-XIX in FIG. 13.

The blood pressure measurement device 1 is an electronic blood pressure measurement device attached to a living body. The present embodiment will be described using an electronic blood pressure measurement device having an aspect of a wearable device attached to a wrist 200 of the living body.

As illustrated in FIGS. 1 to 3, the blood pressure measurement device 1 includes a device body 3, a belt 4 that fixes the device body 3 at the wrist, a curler 5 disposed between the belt 4 and the wrist, a cuff structure 6 including a palm-side cuff 71, a sensing cuff 73, and a back-side cuff 74, and a fluid circuit 7 fluidly connecting the device body 3 and the cuff structure 6.

As illustrated in FIGS. 1 to 5, the device body 3 includes, for example, a case 11, a display unit 12, an operation unit 13, a pump 14, a flow path unit 15, an on-off valve 16, a pressure sensor 17, a power supply unit 18, a vibration motor 19, and a control substrate 20. The device body 3 feeds a fluid to the cuff structure 6 using the pump 14, the on-off valve 16, the pressure sensor 17, the control substrate 20, and the like.

As illustrated in FIGS. 1 to 3, the case 11 includes an outer case 31, a windshield 32 that covers an upper opening of the outer case 31, a base 33 provided at a lower portion of an interior of the outer case 31, and a back lid 35 covering a lower portion of the outer case 31.

The outer case 31 is formed in a cylindrical shape. The outer case 31 includes pairs of lugs 31a provided at respective symmetrical positions in the circumferential direction of an outer circumferential surface, and spring rods 31b each provided between the lugs 31 of each of the two pairs of lugs 31a. The windshield 32 is, for example, a circular glass plate.

The base portion 33 holds the display unit 12, the operation unit 13, the pump 14, the on-off valve 16, the pressure sensor 17, the power supply unit 18, the vibration motor 19, and the control substrate 20. Additionally, the base 33 constitutes a portion of the flow path unit 15 that makes the pump 14 and the cuff structure 6 fluidly continuous.

The back lid 35 covers a living body side end portion of the outer case 31. The back lid 35 is fixed to the living body side end portion of the outer case 31 or the base 33 using, for example, four screws 35a or the like.

The display unit 12 is disposed on the base portion 33 of the outer case 31 and directly below the windshield 32. As illustrated in FIG. 5, the display unit 12 is electrically connected to the control substrate 20. The display unit 12 is, for example, a liquid crystal display or an organic electroluminescence display. The display unit 12 displays various types of information including the date and time and measurement results of blood pressure values such as the systolic blood pressure and diastolic blood pressure, heart rate, and the like.

The operation unit 13 is configured to be capable of receiving an instruction input from a user. For example, the operation unit 13 includes a plurality of buttons 41 provided on the case 11, a sensor 42 that detects operation of the buttons 41, and a touch panel 43 provided on the display unit 12 or the windshield 32, as illustrated in FIG. 5. When operated by the user, the operation unit 13 converts an instruction into an electrical signal. The sensor 42 and the touch panel 43 are electrically connected to the control substrate 20 to output electrical signals to the control substrate 20.

As the plurality of buttons 41, for example, three buttons are provided. The buttons 41 are supported by the base 33 and protrude from the outer circumferential surface of the outer case 31. The plurality of buttons 41 and a plurality of the sensors 42 are supported by the base 33. The touch panel 43 is integrally provided on the windshield 32, for example.

The pump 14 is, for example, a piezoelectric pump. The pump 14 compresses air and feeds compressed air to the cuff structure 6 through the flow path unit 15. The pump 14 is electrically connected to the control substrate 20.

The flow path unit 15 constitutes a flow path connecting from the pump 14 to the palm-side cuff 71 and the back-side cuff 74 and a flow path connecting from the pump 14 to the sensing cuff 73, as illustrated in FIG. 5. Additionally, the flow path unit 15 constitutes a flow path connecting from the palm-side cuff 71 and the back-side cuff 74 to the atmosphere, and a flow path connecting from the sensing cuff 73 to the atmosphere. The flow path unit 15 is a flow path of air constituted by a hollow portion, a groove, a tube, or the like provided in the base portion 33 and the like.

The on-off valve 16 opens and closes a portion of the flow path 15. A plurality of the on-off valves 16 is provided, for example, as illustrated in FIG. 5, and selectively opens and closes the flow path connecting from the pump 14 to the palm-side cuff 71 and the back-side cuff 74, the flow path connecting from the pump 14 to the sensing cuff 73, the flow path connecting from the palm-side cuff 71 and the back-side cuff 74 to the atmosphere, and the flow path connecting from the sensing cuff 73 to the atmosphere, by the combination of opening and closing of each of the on-off valves 16. For example, two on-off valves 16 are used.

The pressure sensor 17 detects the pressures in the palm-side cuff 71, the sensing cuff 73 and the back-side cuff 74. The pressure sensor 17 is electrically connected to the control substrate 20. The pressure sensor 17 converts a detected pressure into an electrical signal, and outputs the electrical signal to the control substrate 20. The pressure sensor 17 is provided in the flow path connecting from the pump 14 to the palm-side cuff 71 and the back-side cuff 74 and in the flow path connecting from the pump 14 to the sensing cuff 73, as illustrated in FIG. 5. These flow paths are continuous through the palm-side cuff 71, the sensing cuff 73, and the back-side cuff 74, and thus the pressure in these flow paths corresponds to the pressure in the internal space of the palm-side cuff 71, the sensing cuff 73, and the back-side cuff 74.

The power supply unit 18 is, for example, a secondary battery such as a lithium ion battery. The power supply unit 18 is electrically connected to the control substrate 20. The power supply unit 18 supplies power to the control substrate 20.

As illustrated in FIGS. 5 and 6, the control substrate 20 includes, for example, a substrate 51, an acceleration sensor 52, a communication unit 53, a storage unit 54, and a control unit 55. The control substrate 20 is constituted by the acceleration sensor 52, the communication unit 53, the storage unit 54, and the control unit 55 that are mounted on the substrate 51.

The substrate 51 is fixed to the base 33 of the case 11 using screws or the like.

The acceleration sensor 52 is, for example, a 3-axis acceleration sensor. The acceleration sensor 52 outputs, to the control unit 55, an acceleration signal representing acceleration of the device body 3 in three directions orthogonal to one another. For example, the acceleration sensor 52 is used to measure, from the detected acceleration, the amount of activity of a living body to which the blood pressure measurement device 1 is attached.

The communication unit 53 is configured to be able to transmit and receive information to and from an external device wirelessly or by wire. For example, the communication unit 53 transmits information controlled by the control unit 55 and information of a measured blood pressure value, a pulse, and the like to an external device via a network, and receives a program or the like for software update from an external device via a network and sends the program or the like to the control unit 55.

In the present embodiment, the network is, for example, the Internet, but is not limited to this. The network may be a network such as a Local Area Network (LAN) provided in a hospital or may be direct communication with an external device using a cable or the like including a terminal of a predetermined standard such as a USB. Thus, the communication unit 53 may be configured to include a plurality of wireless antennas, micro-USB connectors, or the like.

The storage unit 54 pre-stores program data for controlling the overall blood pressure measurement device 1 and a fluid circuit 7, settings data for setting various functions of the blood pressure measurement device 1, calculation data for calculating a blood pressure value and a pulse from pressure measured by the pressure sensors 17, and the like. Additionally, the storage unit 54 stores information such as a measured blood pressure value and a measured pulse.

The control unit 55 is constituted by one or more CPUs, and controls operation of the overall blood pressure measurement device 1 and operation of the fluid circuit. The control unit 55 is electrically connected to and supplies power to the display unit 12, the operation unit 13, the pump 14, each of the on-off valves 16 and the pressure sensors 17. Additionally, the control unit 55 controls operation of the display unit 12, the pump 14, and the on-off valves 16, based on electrical signals output by the operation unit 13 and the pressure sensors 17.

For example, as illustrated in FIG. 5, the control unit 55 includes a main Central Processing Unit (CPU) 56 that controls operation of the overall blood pressure measurement device 1, and a sub-CPU 57 that controls operation of the fluid circuit 7. For example, the main CPU 56 obtains measurement results such as blood pressure values, for example, the systolic blood pressure and the diastolic blood pressure, and the heart rate, from electrical signals output by the pressure sensor 17, and outputs an image signal corresponding to the measurement results to the display unit 12.

For example, the sub-CPU 57 drives the pump 14 and the on-off valves 16 to feed compressed air to the palm-side cuff 71 and the sensing cuff 73 when an instruction to measure the blood pressure is input from the operation unit 13. In addition, the sub-CPU 57 controls driving and stopping of the pump 14 and opening and closing of the on-off valves 16 based on electrical signal output by the pressure sensors 17. The sub-CPU 57 controls the pump 14 and the on-off valves 16 to selectively feed compressed air to the palm-side cuff 71 and the sensing cuff 73 and selectively depressurize the palm-side cuff 71 and the sensing cuff 73.

As illustrated in FIGS. 1 to 3, the belt 4 includes a first belt 61 provided on a first pair of lugs 31a and a first spring rod 31b. and a second belt 62 provided on a second pair of lugs 31a and a second spring rod 31b. The belt 4 is wrapped around the wrist 200 with a curler 5 in between.

As illustrated in FIG. 3, the first belt 61 is referred to as a so-called parent and is configured like a band. The first belt 61 includes a first hole portion 61a provided at a first end portion of the first belt 61 and extending orthogonally to the longitudinal direction of the first belt 61, a second hole portion 61b provided at a second end portion of the first belt 61 and extending orthogonally to the longitudinal direction of the first belt 61, and a buckle 61c provided on the second hole portion 61b. The first hole portion 61a has an inner diameter at which the spring rod 31b can be inserted into the first hole portion 61a and at which the first belt 61 can rotate with respect to the spring rod 31b. In other words, the first belt 61 is rotatably held by the outer case 31 by disposing the first hole portion 61a between the pair of lugs 31a and around the spring rod 31b.

The second hole portion 61b is provided at a tip of the first belt 61. The buckle 61c includes a frame body 61d in a rectangular frame shape and a prong 61e rotatably attached to the frame body 61d. A side of the frame body 61d to which the prong 61e is attached is inserted into the second hole portion 61b. and the frame body 61d is attached in such a manner as to be rotatable with respect to the first belt 61.

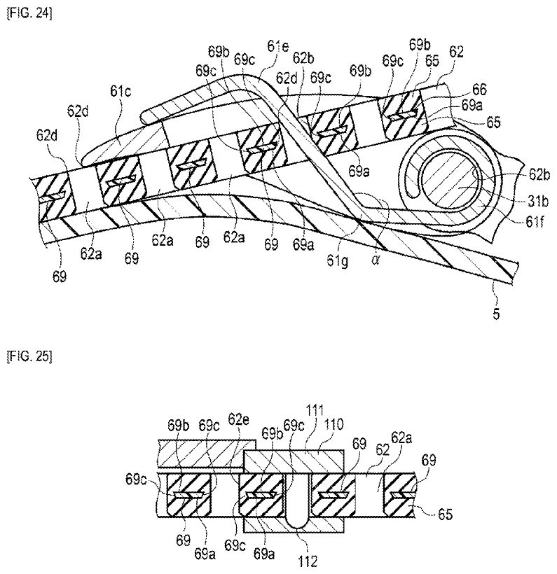

As illustrated in FIGS. 11 and 24, the prong 61e includes a support portion 61f and a rod portion 61g. The support portion 61f is constituted in a ring shape with one side of the frame body 61d rotatably disposed inside the ring.

The rod portion 61g is formed integrally with the support portion 61f. The rod portion 61g is shaped with two bent portions. As a specific example, the rod portion 61g includes a first portion 61g1 continuous with the support portion 61f. a second portion 61g2 continuous with the first portion 61g1 and bent with respect to the first portion 61g1, and a third portion 61g3 continuous with the second portion 61g2 and bent with respect to the second portion 61g2. The first portion 61g1 and the second portion 61g2 constitute one bent portion, and the second portion 61g2 and the third portion 61g3 constitute one bent portion.

The first portion 61g1 is disposed on the inner side of the belt 4 when the first belt 61 is connected to the second belt 62. The first portion 61g1 is constituted in a shape extending in one direction from the support portion 61f.

The second portion 61g2 is disposed in any of small holes 62a in the second belt 62 when the first belt 61 is connected to the second belt 62. The second portion 61g2 is constituted in a shape bending from one end of the first portion 61g1 and extending in a direction intersecting a direction in which the first portion 61g1 extends.

Additionally, the second portion 61g2 has a shape that abut against a wrist 200-side edge of the small hole 62a when the blood pressure measurement device 1 is attached to the wrist 200, and apply, to the edge, a load acting toward a side surface 69c of the second insert 66, which will be described below, of the second belt 62.

Specifically, the angle a formed between the second 61g2 and the first portion 61g1 is set so that the direction of the load applied to the edge of the small hole 62a from the second portion 61g2 is set at an angle corresponding to a direction orthogonal to, or substantially orthogonal to, the side surface 69c of the second insert 66. The angle a is, for example, 120 degrees.

The third portion 61g3 abuts the frame body 61d. The third portion 61g3 is constituted in a shape bending from one end of the second portion 61g2 and extending in a direction intersecting a direction in which the second portion 61g2 extends. In particular, the third portion 61g3 is constituted in a shape extending in a direction intersecting with respect to the second 61g2 in a plane defined by the first portion 61g1 and the second portion 61g2.

The prong 61e configured in this manner is constituted, for example, by performing bending to a rod-shaped member.

As illustrated in FIG. 7, the first belt 61 includes a first belt body 63 and a first insert 64.

The first insert 64 is disposed inside the first belt body 63. The first belt body 63 is constituted by a thermosetting resin, for example. The first belt body 63 is constituted by a flexible resin material that is elastically deformable, for example. One type of thermosetting resin includes, for example, a thermosetting elastomer, and one type of thermosetting elastomer includes, for example, a silicone resin or a fluorine resin.

The first insert 64 is disposed within the first belt body 63. The first insert 64 is constituted to be slightly shorter than the first belt body 63 in the width direction and the circumferential direction, and is covered by the first belt body 63.

The first insert 64 is, for example, a sheet constituted by a material having a higher tensile strength than a resin material constituting the first belt body 63. The tensile strength as used herein indicates the degree of elongation with respect to a tensile load.

Specifically, the material of the first insert 64 is constituted to have a higher tensile strength in the circumferential direction of the living body than the thermosetting resin constituting the first belt body 63. Examples of the material for the first insert 64 include, for example, high strength polyarylate (vectran) fibers, liquid crystal polymers, PET resins, PEN resins, and the like. The first insert 64 is formed like a mesh or film.

As illustrated in FIG. 8, the second belt 62 is referred to as a so-called blade tip, and is constituted in a band-like shape having a width at which the second belt 62 can be inserted into the frame body 61d. In addition, the second belt 62 includes a plurality of small holes 62a into which the prong 61e is inserted. Additionally, the second belt 62 includes a third hole portion 62b provided at first end portion of the second belt 62 and extending orthogonally to the longitudinal direction of the second belt 62. The third hole portion 62b has an inner diameter at which the spring rod 31b can be inserted into the third hole portion 62b and at which the second belt 62 can rotate with respect to the spring rod 31b. In other words, the second belt 62 is rotatably held by the outer case 31 by disposing the third hole portion 62b between the pair of lugs 31a and around the spring rod 31b.

As illustrated in FIG. 9, the second belt 62 includes a second belt body 65 and a second insert 66.

The second belt body 65 includes a second insert 66 disposed inside the second belt body 65. The second belt body 65 is constituted by a thermosetting resin, for example. The second belt body 65 is constituted by the same material as that of the first belt body 63 of the first belt 61 described above.

The second insert 66 is constituted to have a length in the width direction and a length in the circumferential direction being shorter than those of the second belt body 65, and is covered by the second belt body 65. The second insert 66 is disposed in the second belt body 65. The second insert 66 is disposed in the center of the second belt 62 in a thickness direction. A third hole portion 62b. for example, is formed at a longitudinal end portion of the second insert 66.

The second insert 66 is constituted by a thin plate-like member. A material constituting the second insert 66 has a higher tensile strength than a material constituting the second belt body 65. The tensile strength indicates the degree of elongation with respect to the tensile load. The second insert 66 is constituted by the same material as that of the first insert 64 of the first belt 61 described above, for example.

As illustrated in FIG. 10, a fitting portion 67a into which a positioning pin is fitted is formed in one main surface 67 of the second insert 66. The fitting portion 67a performs positioning the second insert 66 relative to a mold when the mold is used to form the second belt 62 using the second insert 66 and the resin material. The fitting portion 67a is a hole penetrating the second insert 66, as an example. Note that the fitting portion 67a is not limited to being constituted as a hole. The fitting portion 67a may be a recessed portion. The fitting portion 67a is formed at a position offset from the center with respect to the left-right direction of the second insert 66. Note that the configuration of the second insert 66 is configured in a left-right symmetric shape except the fitting portion 67a. Here, the left-right symmetry refers to being symmetry about a center line that extends through the center of the second insert 66 in the width direction and is parallel to the longitudinal direction of the second insert 66. In other words, the second insert 66 includes the fitting portion 67a. and thus the second insert 66 has a left-right asymmetric shape.

The second insert 66 also includes a plurality of holes 68 with the small holes 62a disposed on the inner side of the respective holes 68. Each of the plurality of holes 68 is penetrating through the second insert 66 in the thickness direction. Each of the holes 68 is constituted to have a shape larger than each of the small hole 62a.

As illustrated in FIG. 24, portions of the second insert 66 between the adjacent holes 68 are disposed in the second belt body 65. Here, the portion of the second insert 66 between the adjacent holes 68 is referred to as a hole reinforcing portion 69. The hole reinforcing portion 69 is disposed, for example, in the center of the second belt 62 in the thickness direction. Specifically, the center of the hole reinforcing portion 69 in the thickness direction coincides with the center of the second belt body 65 in the thickness direction.

The hole reinforcing portion 69 is disposed between adjacent small holes 62a. Specifically, the center of the hole reinforcing portion 69 is disposed in the center of a portion between two adjacent small holes 62a. In addition, the length of the hole reinforcing portion 69 along the longitudinal direction of the second belt 62 is a sufficient length such that the hole reinforcing portion 69 is prevented from being deformed during usage of the blood pressure measurement device 1.

Note that a length L1 between two adjacent small hole holes 62a in the second belt 62 along the longitudinal direction of the second belt 62 is set to be a length such that the belt 4 can be attached to wrists 200 of various users at an appropriate tightening force and such that the strength of the portion between two adjacent small holes 62a in the second belt 62 can be set to prevent the portion from being subjected to deformation such as elongation due to blood pressure measurement.

The length L1 between two adjacent small holes 62a along the longitudinal direction of the second belt 62, as used herein, refers to the shortest length between the edge of one small hole 62a and the edge of the other small hole 62a. which are opposite each other, along the longitudinal direction of the second belt 62. In other words, the length L1 is the shortest length of the portion of the second belt 62 sandwiched between the two adjacent small holes 62a along the longitudinal direction of the second belt 62. In the present embodiment, as an example, the small hole 62a is formed in a long hole shape that is long in the width direction orthogonal to the longitudinal direction of the second belt 62, and is constituted to include two edges being parallel to the width direction. Thus, in the present embodiment, as illustrated in FIG. 9, the length L1 between the two adjacent small holes 62a along the longitudinal direction of the second belt 62 is the length along the longitudinal direction of the second belt 62 between the edges of the two adjacent small holes 62a along the width direction. The optimal value of the length L1 between two adjacent small holes 62a along the longitudinal direction of the second belt 62 is 4 mm.

Additionally, the appropriate tightening force as used herein is the tightening force that allows the blood pressure to be accurately measured. By setting, to 4 mm, the length L1 between the two adjacent small holes 62a along the longitudinal direction of the second belt 62, the tightening force applied to the wrist 200 by the belt 4 can be precisely adjusted. Thus, although the circumferential length of the wrist 200 varies depending on the user, by setting, to 4 mm, the length L1 between the two adjacent small holes 62a along the longitudinal direction of the second belt 62, the small hole 62a into which the prong 61e is inserted can be selected, to generate a tightening force for each of the wrists 200 of various users enabling accurate blood pressure measurement.

Furthermore, by setting, to 4 mm, the length L1 between the two adjacent small holes 62a along the longitudinal direction of the second belt 62, the strength of the portion between the two adjacent small holes 62a in the second belt 62 can be set to be a strength such that the portion can be prevented from being deformed against a load input from the prong 61e due to blood pressure measurement. Thus, even for repeated blood pressure measurements, the small holes 62a are prevented from being deformed, enabling accurate blood pressure measurements.

In addition, a cross section of the hole reinforcing portion 69 orthogonal to the width direction of the second belt 62 is constituted like a trapezoid. In other words, the hole reinforcing portion 69 includes a first surface 69a facing one main surface of the second belt 62, a second surface 69b facing the other main surface of the second belt 62, and two side surfaces 69c continuous with the first surface 69a and the second surface 69b. As illustrated in FIG. 24, the first face 69a is disposed on the wrist 200 side with respect to the second face 69b when the blood pressure measurement device 1 is attached to the wrist 200.

The length of the first surface 69a along the longitudinal direction of the second belt 62 is smaller than the length of the second surface 69b along the longitudinal direction of the second belt. Both side surfaces 69c are inclined surfaces that are inclined with respect to the first surface 69a and the second surface 69b. The angle formed by the first surface 69a and one of the side surfaces 69c and the angle formed by the first surface 69a and the other side surface 69c are, for example, obtuse. The angle formed by the second surface 69b and one of the side surfaces 69c and the angle formed by the second surface 69b and the other side surface 69c are, for example, acute.

The thickness of the second belt body 65 between the hole 68 and the small hole 62a. in other words, the length between the hole 68 and the small hole 62a along the longitudinal direction of the second belt 62 of the second belt body 65 corresponds to a thickness sufficient to suppress cracking of the surface of the second belt 62 due to the load from the prong 61e input during repeated use of the blood pressure measurement device 1.

In other words, the portion between the small hole 62a and the side surface 69c along the longitudinal direction of the second belt 62 deforms due to the load input from the prong 61e when the blood pressure measurement device 1 is in use, but an increased thickness of this portion correspondingly increases a deformation margin, making the deformation more significant. As a result, the deformation may cause the outer surface of the second belt 62 to be more significantly cracked. In the present embodiment, the thickness between the small hole 62a and the hole 68 along the longitudinal direction of the second belt 62 is constituted to be a thickness sufficient to reduce deformation of the portion between the small hole 62a and the hole 68 along the longitudinal direction of the second belt 62, allowing suppression of cracking of the outer surface of the second belt 62 resulting from deformation generated by a load input from the prong 61e in association with repeated use of the blood pressure measurement device 1.

The second insert 66 configured as described above is constituted by performing stamping using pressing on a thin plate-like member. In the case where the second insert 66 is formed by stamping using pressing, a cross section of the hole reinforcing portion 69 is constituted like a trapezoid in which, compared to the width of a surface which is on a side where a mold is pressed, the width of the other surface is larger. Note that the surface which is on the side where the mold is pressed, as used herein, is a surface constituting the second surface 69b. The width of the surface which is on the side where the mold is pressed refers to the length along the longitudinal direction of the second belt 62. In other words, by forming the second insert 66 by stamping using pressing, the cross-section of the hole reinforcing portion 69 is constituted like a trapezoid.

Now, description will be given of an example of a method for manufacturing the belt 4, which is a part of the method for manufacturing the blood pressure measurement device 1 according to one embodiment. A method for the second belt 62 will be described as a representative.

In the manufacturing method of the present embodiment, as an example, first, a primary molded article 62A is formed by primary molding, and then the second belt 62 is completed by secondary molding. First, as illustrated in step ST11 in FIG. 12, the primary molded article 62A is formed. The primary molded article 62A includes a base 65a and a second insert 66. The base portion 65a constitutes one side portion of the second belt body 65 in the thickness direction of the second belt 62 across the second insert 66. The one side portion corresponds to, as an example, the wrist 200 side when the blood pressure measurement device 1 is attached to the wrist 200. The primary molded article 62A is formed using a first mold 221 and a second mold 222.

The second insert 66 is placed on the first mold 221. The first mold 221 includes a first pin 221a and a plurality of second pins 221b.

The first pin 221a is constituted in a shape that fits into the fitting portion 67a of the second insert 66. The length of the first pin 221a along a protrusion direction of the first pin 221a is larger than the thickness of the second insert 66, as an example.

A part of each of the plurality of second pins 221b is constituted in a shape that fits into the hole 68 in the second insert 66. Additionally, another part of each of the plurality of second pins 221b is constituted in a shape that forms the small hole 62a in the base portion 65a. The second mold 222 constitutes, between the first mold 221 and the second insert 66, a cavity corresponding to the base 65a.

In molding the primary molded article 62A, first, the second insert 66 is placed on the first mold 221. At this time, the first pin 221a is fitted into the fitting portion 67a of the second insert 66, and the second pins 221b are fitted into the holes 68. At this time, the second insert 66 is placed on the first mold 221 with the first surface 69a of the hole reinforcing portion 69 oriented to face the cavity side.

The first pin 221a is fitted into the fitting portion 67a to position the second insert 66 relative to the first mold 221. In this configuration, the fitting portion 67a is disposed at a position offset from the left-right center of the second insert 66, and thus the second insert 66 is positioned in the correct orientation on the first mold 221 in a state in which the first pin 221a is fitted into the fitting portion 67a. A tip portion of first pin 221a protrudes out of the second insert 66. Then, the second mold 222 is overlaid on the first mold 221. Then, the cavity between the second mold 222 and both the first mold 221 and the second insert 66 is filled with resin, for example, by injection. Then, the primary molded article 62A is removed from the first mold 221 and the second mold 222. In the primary molded article 62A constituted as described above, the first surface 69a of the hole reinforcing portion 69 faces the base portion 65a.

Then, as illustrated in step ST12 in FIG. 12, a third and a fourth molds 223 and 224 are used to form the second belt 62 from the primary molded article 62A. Specifically, the third mold 223 includes a recess 223a into which the primary molded article 62A is fitted and third pins 223b that form the small holes 62a. The fourth mold 224 constitutes a cavity corresponding to the remaining portion of the second belt body 65 between the fourth mold 224 and the primary molded article 62A installed in the third mold 223.

First, the primary molded article 62A is disposed in the concave portion 223a of the third mold 223. At this time, the primary molded article 62A is disposed with the base portion 65a oriented to face an inner surface of the recessed portion 223a and with the second insert 66 oriented to face outward, and the third pin 223b is fitted into the small hole 62a in the base portion 65a. Then, the fourth mold 224 is overlaid on the third mold 223. The cavity between the primary molded article 62A and the fourth mold 224 is then filled with resin, for example, by injection.

As described above, the second belt 62 is completed by steps ST11 and ST12. A method for forming the first belt 61 may be similar to the method for forming the second belt 62.

Thus, with the second belt 62 inserted into the frame body 61d and with the prong 61e inserted into the small hole 62a. the first belt 61 and the second belt 62 are integrally connected together, and the belt 4 forms, along with the outer case 31, a ring shape following the wrist 200 along the circumferential direction.

As illustrated in FIG. 4, the curler 5 is configured in a band-like shape that curves in such a manner as to follow along the circumferential direction of the wrist. The curler 5 is formed with a first end and a second end spaced apart from each other. For example, a first end-side outer surface of the curler 5 is fixed to the back lid 35 of the device body 3. The first end and the second end of the curler 5 are disposed at positions where the first end and the second end protrude from the back lid 35. Furthermore, the first end and the second end of the curler 5 are located adjacent to each other at a predetermined distance from each other.

As a specific example, the curler 5 is fixed to a living body side end portion of the outer case 31 or the base 33 along with the back lid 35 using screws 35a or the like. Additionally, the curler 5 is fixed to the back lid 35 such that the first end and the second end are located on one lateral side of the wrist 200 when the blood pressure measurement device 1 is attached to the wrist 200.

As a specific example, as illustrated in FIG. 1, FIG. 2, and FIG. 4, the curler 5 has a shape that curves along a direction orthogonal to the circumferential direction of the wrist, in other words, along the circumferential direction of the wrist 200 in a side view from the longitudinal direction of the wrist. The curler 5 extends, for example, from the device body 3 through the hand back side of the wrist 200 and one lateral side of the wrist 200 to the hand palm side of the wrist 200 and toward the other lateral side of the wrist 200. Specifically, by curving along the circumferential direction of the wrist 200, the curler 5 is disposed across the most of the wrist 200 in the circumferential direction, with both ends of the curler 5 spaced at a predetermined distance from each other.

The curler 5 has hardness appropriate to provide flexibility and shape retainability. Here, “flexibility” refers to deformation of the shape of the curler 5 in a radial direction at the time of application of an external force of the belt 4 to the curler 5. For example, “flexibility” refers to deformation of the shape of the curler 5 in a side view in which the curler 5 approaches the wrist, is along the shape of the wrist, or follows to the shape of the wrist when the curler 5 is pressed by the belt 4. Furthermore, “shape retainability” refers to the ability of the curler 5 to maintain a pre-imparted shape when no external force is applied to the curler 5. For example, “shape retainability” refers to, in the present embodiment, the ability of the curler 5 to maintain the shape in a shape curving along the circumferential direction of the wrist.

The cuff structure 6 is disposed on an inner circumferential surface of the curler 5, and is held along the shape of the inner circumferential surface of the curler 5. As a specific example, the cuff structure 6 is fixed to the curler 5 by disposing the palm-side cuff 71 and the back-side cuff 74 on the inner circumferential surface of the curler 5, and bonding the palm-side cuff 71 and the back-side cuff 74 to an outer circumferential surface or the inner circumferential surface of the curler 5 with a double-sided tape, an adhesive, or the like. In the present embodiment, the palm-side cuff 71 and the back-side cuff are bonded to the inner circumferential surface of the curler 5 with a double-sided tape, an adhesive, or the like.

The curler 5 is constituted by a resin material. Furthermore, a material that is harder than the palm-side cuff 71 and the back-side cuff 74 is used for the curler 5. The curler 5 is formed, for example, to a thickness of approximately 1 mm.

As illustrated in FIGS. 1 to 4 and FIGS. 13 to 19, the cuff structure 6 includes the palm-side cuff 71, the back plate 72, the sensing cuff 73, and the back-side cuff 74. The cuff structure 6 is fixed to the curler 5. The cuff structure 6 includes the palm-side cuff 71, the back plate 72, and the sensing cuff 73 that are stacked one another and disposed on the curler 5, and the back-side cuff 74 that is spaced apart from the palm-side cuff 71, the back plate 72, and the sensing cuff 73 and disposed on the curler 5.

As a specific example, the cuff structure 6 includes the palm-side cuff 71, the back plate 72, the sensing cuff 73, and the back-side cuff 74 that are disposed on an inner surface of the curler 5. The cuff structure 6 is fixed to the inner surface of the curler 5 on the hand palm side of the wrist 200 with the palm-side cuff 71, the back plate 72, and the sensing cuff 73 stacked in this order from the inner surface of the curler 5 toward the living body. In addition, the cuff structure 6 includes the back-side cuff 74 disposed on the inner surface of the curler 5 on the hand back side of the wrist 200. Each of the members of the cuff structure 6 is fixed to an adjacent member of the cuff structure 6 in a stacking direction with a double-sided tape, an adhesive, or the like.

The palm-side cuff 71 is a so-called pressing cuff. The palm-side cuff 71 is fluidly connected to the pump 14 through the flow path unit 15. The palm-side cuff 71 is inflated to press the back plate 72 and the sensing cuff 73 toward the living body side. The palm-side cuff 71 includes air bags 81 in a plurality of, for example, two layers.

Here, the air bags 81 are bag-like structures, and in the present embodiment, the blood pressure measurement device 1 is configured to use air with the pump 14, and thus the present embodiment will be described using the air bags. However, in a case where a fluid other than air is used, the bag-like structures may be fluid bags such as liquid bags. The plurality of air bags 81 are stacked and are in fluid communication with one another in the stacking direction.

Each of the air bags 81 is constituted in a rectangular shape that is long in one direction. The air bag 81 is constituted, for example, by combining two sheet members 86 that are long in one direction, and thermally welding edges of the sheet members. As a specific example, as illustrated in FIGS. 13 to 15, the two-layer air bags 81 include a first sheet member 86a. a second sheet member 86b. a third sheet member 86c. and a fourth sheet member 86d in this order from the living body side. The second sheet member 86b constitutes a first-layer air bag 81 along with the first sheet member 86a. the third sheet member 86c is integrally bonded to the second sheet member 86b. and the fourth sheet member 86d constitutes a second-layer air bag 81 along with the third sheet member 86c. Note that the two-layer air bags 81 are integrally constituted by joining each of the sheet members 86 of the adjacent air bags 81 by bonding with a double-sided tape, an adhesive, or the like, or welding or the like.

Edge portions of four sides of the first sheet member 86a are welded to corresponding edge portions of four sides of the second sheet member 86b to constitute the air bag 81. The second sheet member 86b and the third sheet member 86c are disposed facing each other, and each includes a plurality of openings 86b1 and 86c1 through which the two air bags 81 are fluidly continuous. The fourth sheet member 86d is disposed on the curler 5 and is bonded to the inner circumferential surface or the outer circumferential surface of the curler 5 with a double-sided tape, an adhesive, or the like.

Edge portions of four sides of the third sheet member 86c are welded to corresponding edge portions of four sides of the fourth sheet member 86d to constitute the air bag 81.

The back plate 72 is applied to an outer surface of the first sheet member 86a of the palm-side cuff 71 with an adhesive layer, a double-sided tape, or the like. The back plate 72 is formed in a plate shape using a resin material. The back plate 72 is made of polypropylene, for example, and is formed into a plate shape having a thickness of approximately 1 mm. The back plate 72 has shape followability.

Here, “shape followability” refers to a function of the backplate 72 by which the back plate 72 can be deformed in such a manner as to follow the shape of a contacted portion of the wrist 200 to be disposed, the contacted portion of the wrist 200 refers to a region of the wrist 200 that is faced by the back plate 72, and the contact as used herein includes both direct contact and indirect contact with the sensing cuff 73 in between.

For example, as illustrated in FIG. 15, the back plate 72 includes a plurality of grooves 72a formed in both main surfaces of the back plate 72 and extending in a direction orthogonal to the longitudinal direction. As illustrated in FIG. 15, a plurality of the grooves 72a are provided in both main surfaces of the back plate 72. The plurality of grooves 72a provided in one of the main surfaces face the corresponding grooves 72a provided in the other main surface in the thickness direction of the back plate 72. Additionally, the plurality of grooves 72a are disposed at equal intervals in the longitudinal direction of the back plate 72.

In the back plate 72, portions including the plurality of grooves 72a are thinner than portions including no grooves 72a and thus the portions including the plurality of grooves 72a are easily deformed. Accordingly, the back plate 72 has shape followability of deforming in such a manner as to follow to the shape of the wrist 200 and extending in the circumferential direction of the wrist. The back plate 72 is formed such that the length of the back plate 72 is sufficient to cover the hand palm side of the wrist 200. The back plate 72 transfers the pressing force from the palm-side cuff 71 to the back plate 72 side main surface of the sensing cuff 73 in a state in which the back plate 72 is extending along the shape of the wrist 200.

The sensing cuff 73 is fixed to the living body side main surface of the back plate 72. The sensing cuff 73 is in direct contact with a region of the wrist 200 where an artery 210 resides, as illustrated in FIGS. 18 and 19. The artery 210 as used herein is the radial artery and the ulnar artery. The sensing cuff 73 is formed in the same shape as that of the back plate 72 or a shape that is smaller than that of the back plate 72, in the longitudinal direction and the width direction of the back plate 72. The sensing cuff 73 is inflated to compress a hand palm-side region of the wrist 200 in which the artery 210 resides. The sensing cuff 73 is pressed by the inflated palm-side cuff 71 toward the living body side with the back plate 72 in between.

As a specific example, the sensing cuff 73 includes one air bag 91, a tube 92 that communicates with the air bag 91, and a connection portion 93 provided at a tip of the tube 92. One main surface of the air bag 91 of the sensing cuff 73 is fixed to the back plate 72. For example, the sensing cuff 73 is applied to the living body side main surface of the back plate 72 using a double-sided tape, an adhesive layer, or the like.

Here, the air bag 91 is a bag-like structure, and in the present embodiment, the blood pressure measurement device 1 is configured to use air with the pump 14, and thus the present embodiment will be described using the air bag. However, in a case where a fluid other than air is used, the bag-like structure may be a liquid bag and the like.

The air bag 91 is constituted in a rectangular shape that is long in one direction. The air bag 91 is constituted, for example, by combining two sheet members 96 that are long in one direction, and thermally welding edges of the sheet members. As a specific example, the air bag 91 includes a fifth sheet member 96a and a sixth sheet member 96b in this order from the living body side as illustrated in FIGS. 9 and 13.

For example, the fifth sheet member 96a and the sixth sheet member 96b are fixed by welding, with a tube 92 that is fluidly continuous with the internal space of the air bag 91 being disposed on one side of each of the fifth sheet member 96a and the sixth sheet member 96b. For example, the fifth sheet member 96a and the sixth sheet member 96b are welded together integrally with the tube 92 by welding edge portions of four sides of the fifth sheet member 96a to corresponding edge portions of four sides of the sixth sheet member 96b in a state in which the tube 92 is disposed between the fifth sheet member 96a and the sixth sheet member 96b.

The tube 92 is provided at one longitudinal end portion of the air bag 91. As a specific example, the tube 92 is provided at an end portion of the air bag 91 near the device body 3. The tube 92 includes the connection portion 93 at the tip. The tube 92 is connected to the flow path unit 15 and constitutes a flow path between the device body 3 and the air bag 91. The connection portion 93 is connected to the flow path unit 15. The connection portion 93 is, for example, a nipple.

The back-side cuff 74 is a so-called tensile cuff. The back-side cuff 74 is fluidly connected to the pump 14 through the flow path unit 15. The back-side cuff 74 is inflated to press the curler 5 such that the curler 5 is spaced apart from the wrist 200, pulling the belt 4 and the curler 5 toward the hand back side of the wrist 200. The back-side cuff 74 includes air bags 101 including a plurality of, for example, six layers, a tube 102 in communication with the air bags 101, and a connection portion 103 provided at a tip of the tube 102.

Additionally, the back-side cuff 74 is configured such that the thickness of the back-side cuff 74 in an inflating direction, in the present embodiment, in the direction in which the curler 5 and the wrist 200 face each other, during inflation, is larger than the thickness of the palm-side cuff 71 in the inflating direction during inflation and than the thickness of the sensing cuff 73 in the inflating direction during inflation. Specifically, the air bags 101 of the back-side cuff 74 include more layers than the air bags 81 in the palm-side cuff 71 and the air bag 91 in the sensing cuff 73, and are thicker than the palm-side cuff 71 and the sensing cuff 73 when the air bags 101 are inflated from the curler 5 toward the wrist 200.

Here, the air bag 101 is a bag-like structure, and in the present embodiment, the blood pressure measurement device 1 is configured to use air with the pump 14, and thus the present embodiment will be described using the air bag. However, in a case where a fluid other than air is used, the bag-like structure may be a fluid bag such as a liquid bag. A plurality of the air bags 101 are stacked and are in fluid communication in the stacking direction.

The air bag 101 is constituted in a rectangular shape that is long in one direction. The air bag 101 is constituted, for example, by combining two sheet members 106 that are long in one direction, and thermally welding edges of the sheet members. As a specific example, as illustrated in FIGS. 16 and 17, the six-layer air bags 101 include a seventh sheet member 106a. an eighth sheet member 106b. a ninth sheet member 106c. a tenth sheet member 106d. an eleventh sheet member 106e. a twelfth sheet member 106f. a thirteenth sheet member 106g. a fourteenth sheet member 106h. a fifteenth sheet member 106i. a sixteenth sheet member 106j. a seventeenth sheet member 106k. and an eighteenth sheet member 1061 in this order from the living body side. Note that the six-layer air bags 101 are integrally constituted by joining each of the sheet members 106 of the adjacent air bags 101 by bonding with a double-sided tape, an adhesive, or the like, or welding or the like.

Edge portions of four sides of the seventh sheet member 106a are welded to corresponding edge portions of four sides of the eighth sheet member 106b to constitute a first-layer air bag 101. The eighth sheet member 106b and the ninth sheet member 106c are disposed facing each other and are integrally bonded together. The eighth sheet member 106b and the ninth sheet member 106c include a plurality of openings 106b1 and 106c1 through which the adjacent air bags 101 are fluidly continuous. Edge portions of four sides of the ninth sheet member 106c are welded to corresponding edge portions of four sides of the tenth sheet member 106d to constitute a second-layer air bag 101.

The tenth sheet member 106d and the eleventh sheet member 106e are disposed facing each other and are integrally bonded together. The tenth sheet member 106d and the eleventh sheet member 106e include a plurality of openings 106d1 and 106e1 through which the adjacent air bags 101 are fluidly continuous. Edge portions of four sides of the eleventh sheet member 106e are welded to corresponding edge portions of four sides of the twelfth sheet member 106f to constitute a third-layer air bag 101.

The twelfth sheet member 106f and the thirteenth sheet member 106g are disposed facing each other and are integrally bonded together. The twelfth sheet member 106f and the thirteenth sheet member 106g include a plurality of openings 106f1 and 106g1 through which the adjacent air bags 101 are fluidly continuous. Edge portions of four sides of the thirteenth sheet member 106g are welded to corresponding edge portions of four sides of the fourteenth sheet member 106h to constitute a fourth-layer air bag 101.

The fourteenth sheet member 106h and the fifteenth sheet member 106i are disposed facing each other and are integrally bonded together. The fourteenth sheet member 106h and the fifteenth sheet member 106i include a plurality of openings 106h1 and 106i1 through which the adjacent air bags 101 are fluidly continuous. Edge portions of four sides of the fifteenth sheet member 106i are welded to corresponding edge portions of four sides of the sixteenth sheet member 106j to constitute a fifth-layer air bag 101.

The sixteenth sheet member 106j and the seventeenth sheet member 106k are disposed facing each other and are integrally bonded together. The sixteenth sheet member 106j and the seventeenth sheet member 106k include a plurality of openings 106j1 and 106k1 through which the adjacent air bags 101 are fluidly continuous. Edge portions of four sides of the seventeenth sheet member 106k are welded to corresponding edge portions of four sides of the eighteenth sheet member 106l to constitute a sixth-layer air bag 101. In addition, for example, a tube 102 that is fluidly continuous with the internal space of the air bag 101 is disposed on one side of the seventeenth sheet member 106k and the eighteenth sheet member 106l, and is fixed by welding. For example, in a state in which the tube 102 is disposed between the seventeenth sheet member 106k and the eighteenth sheet member 106l, the edge portions of the seventeenth sheet member 106k are welded to the edge portions of the eighteenth sheet member 106l in a rectangular frame shape to form the air bag 101. Thus, the tube 102 is integrally welded to the air bag 101.

For example, the sixth-layer air bag 101 as described above is constituted integrally with the second layer air bag 81 of the palm-side cuff 71. Specifically, the seventeenth sheet member 106k is constituted integrally with the third sheet member 86c. and the eighteenth sheet member 1061 is constituted integrally with the fourth sheet member 86d.

In more detail, the third sheet member 86c and the seventeenth sheet member 106k constitute a rectangular sheet member that is long in one direction, and the eighteenth sheet member 1061 and the fourth sheet member 86d constitute a rectangular sheet member that is long in one direction. Then, these sheet members are stacked one another, and welding is performed such that first end portion side is welded in a rectangular frame shape, whereas a part of one side on the second end portion side is not welded. Thus, the second-layer air bag 81 of the palm-side cuff 71 is constituted. Then, welding is performed such that the second end portion side is welded in a rectangular frame shape, whereas a part of one side on the first end portion side is not welded. Thus, the sixth-layer air bag 101 in the back-side cuff 74 is constituted. In addition, a part of one side on the facing side of each of the second-layer air bag 81 and the sixth-layer air bag 101 is not welded, and thus the second-layer air bag 81 and the sixth-layer air bag 101 are fluidly continuous.

The tube 102 is connected to one air bag 101 of the six-layer air bags 101 and is provided at one longitudinal end portion of the air bag 101. As a specific example, the tube 102 is provided on the curler 5 side of the six-layer air bags 101 and is provided at the end portion close to the device body 3. The tube 102 includes a connection portion 103 at the tip. The tube 102 constitutes a flow path included in the fluid circuit 7 and located between the device body 3 and the air bags 101. The connection portion 103 is, for example, a nipple.

Note that, as described above, in the present embodiment, the configuration has been described in which a part of the back-side cuff 74 is constituted integrally with the palm-side cuff 71 and is fluidly continuous with the palm-side cuff 71. However, no such limitation is intended. For example, as illustrated in FIG. 14, the back-side cuff 74 may be constituted separately from the palm-cuff 71 and may be fluidly discontinuous with the palm-cuff 71. For such a configuration, the palm-side cuff 71 may be configured such that, like the sensing cuff 73 and the back-side cuff 74, the palm-side cuff 71 is further provided with a tube and a connection portion, and in the fluid circuit 7 as well, the palm-side cuff 71 is connected to a flow path through which the fluid is fed to the palm-side cuff 71, a check valve, and a pressure sensor.

Additionally, the sheet members 86, 96, and 106 forming the palm-side cuff 71, the sensing cuff 73, and the back-side cuff 74 are formed of a thermoplastic elastomer. Examples of thermoplastic elastomer constituting the sheet members 86, 96, and 106 include thermoplastic polyurethane based resin (hereinafter referred to as TPU), vinyl chloride resin, ethylene-vinyl acetate resin, thermoplastic polystyrene based resin, thermoplastic polyolefin resin, thermoplastic polyester based resin, and thermoplastic polyamide resin.

For example, the sheet members 86, 96, and 106 are formed using a molding method such as T-die extrusion molding or injection molding. After being molded by each molding method, the sheet members 86, 96, and 106 are sized into predetermined shapes, and the sized individual pieces are joined by welding or the like to constitute bag-like structures 81, 91, and 101. A high frequency welder or laser welding is used as the welding method.

The fluid circuit 7 is constituted by the case 11, the pump 14, the flow path unit 15, the on-off valves 16, the pressure sensors 17, the palm-side cuff 71, the sensing cuff 73, and the back-side cuff 74. A specific example of the fluid circuit 7 will be described below with two on-off valves 16 that are used in the fluid circuit 7 being designated as a first on-off valve 16A and a second on-off valve 16B, and two pressure sensors 17 that are used in the fluid circuit 17 being designated as a first pressure sensor 17A and a second pressure sensor 17B.

As illustrated in FIG. 5, the fluid circuit 7 includes, for example, a first flow path 7a that makes the palm-side cuff 71 and the back-side cuff 74 continuous with the pump 14, a second flow path 7b constituted by branching from a middle portion of the first flow path 7a and making the sensing cuff 73 continuous with the pump 14, and a third flow path 7c connecting the first flow path 7a to the atmosphere. Additionally, the first flow path 7a includes the first pressure sensor 17A. The first on-off valve 16A is provided between the first flow path 7a and the second flow path 7b. The second flow path 7b includes a second pressure sensor 17B. The second on-off valve 16B is provided between the first flow path 7a and the third flow path 7c.

In the fluid circuit 7 as described above, the first on-off valve 16A and the second on-off valve 16B are closed to connect only the first flow path 7a to the pump 14, and the pump 14 and the palm-side cuff 71 are fluidly connected. In the fluid circuit 7, the first on-off valve 16A is opened and the second on-off valve 16B is closed to connect the first flow path 7a and the second flow path 7b. thus fluidly connecting the pump 14 and the back-side cuff 74, the back-side cuff 74 and the palm-side cuff 71, and the pump 14 and the sensing cuff 73. In the fluid circuit 7, the first on-off valve 16A is closed and the second on-off valve 16B is opened to connect the first flow path 7a and the third flow path 7c. fluidly connecting the palm-side cuff 71, the back-side cuff 74, and the atmosphere together. In the fluid circuit 7, the first on-off valve 16A and the second on-off valve 16B are opened to connect the first flow path 7a. the second flow path 7b. and the third flow path 7c. fluidly connecting the palm-side cuff 71, the sensing cuff 73, the back-side cuff 74, and the atmosphere together.

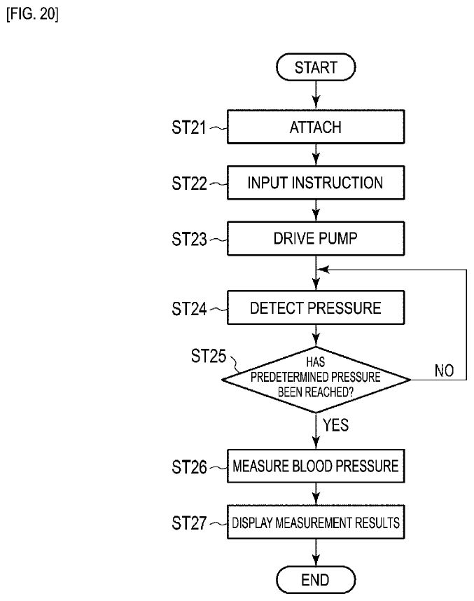

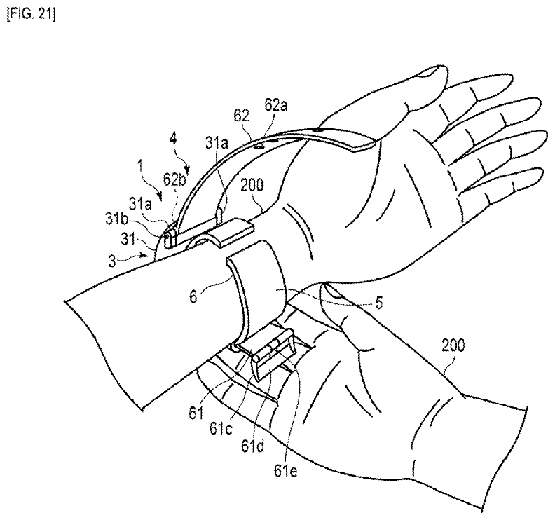

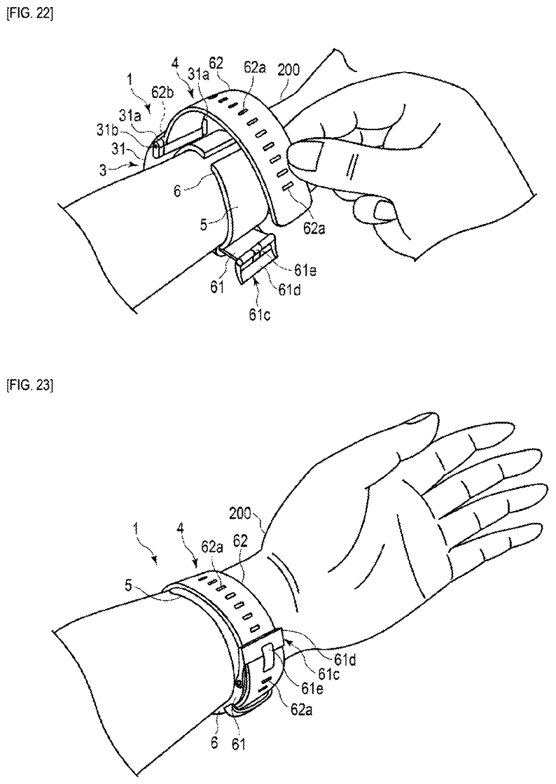

Next, an example of measurement of a blood pressure value using the blood pressure measurement device 1 will be described using FIGS. 20 to 23. FIG. 22 is a flowchart illustrating an example of a blood pressure measurement using the blood pressure measurement device 1, illustrating both an operation of a user and an operation of the control unit 55. Additionally, FIGS. 21 to 23 illustrate an example of the user wearing the blood pressure measurement device 1 on the wrist 200.

First, the user attaches the blood pressure measurement device 1 to the wrist 200 (step ST21). As a specific example, for example, the user inserts one of the wrists 200 into the curler 5, as illustrated in FIG. 21.

At this time, in the blood pressure measurement device 1, the device body 3 and the sensing cuff 73 are disposed at opposite positions in the curler 5, and thus the sensing cuff 73 is disposed in a region on the hand palm side of the wrist 200 in which the artery 210 resides. Thus, the device body 3 and the back-side cuff 74 are disposed on the hand back side of the wrist 200. Then, as illustrated in FIG. 22, the user passes the second belt 62 through the frame body 61d of the buckle 61c of the first belt 61 with the hand opposite to the hand on which the blood pressure measurement device 1 is disposed. The user then pulls the second belt 62 to bring the member on the inner circumferential surface side of the curler 5, that is, the cuff structure 6, into close contact with the wrist 200, and inserts the prong 61e into the small hole 62a. Thus, as illustrated in FIG. 23, the first belt 61 and the second belt 62 are connected, and the blood pressure measurement device 1 is attached to the wrist 200.

Thus, when the blood pressure measurement device 1 is attached to the wrist 200, the hole reinforcing portion 69 of the second insert 66 of the second belt 62 is disposed such that the first surface 69a. which has a smaller length along the longitudinal direction of the second belt 62, is placed on the wrist 200 side with respect to the second surface 69b. which has a larger length along the longitudinal direction of the second belt 62.

Furthermore, the second belt 62 curves in such a manner as to follow to the shape of the wrist 200. As illustrated in FIG. 24, a part of the second portion 61g2 of the prong 61e abuts against a portion of the wrist 200-side edge of the small hole 62a. at which the prong 61e is disposed, the portion being located on the support portion 61f-side in the longitudinal direction of the second belt 62.

Then, the user operates the operation unit 13 to input an instruction corresponding to the start of measurement of the blood pressure value. The operation unit 13, on which an input operation of the instruction has been performed, outputs an electrical signal corresponding to the start of the measurement to the control unit 55 (step ST22). The control unit 55 receives the electrical signal, and then for example, opens the first on-off valve 16A, closes the second on-off valve 16B, and drives the pump 14 to feed compressed air to the palm-side cuff 71, the sensing cuff 73, and the back-side cuff 74 through the first flow path 7a and the second flow path 7b (step ST23). Thus, the palm-side cuff 71, the sensing cuff 73, and the back-side cuff 74 start to be inflated.

The first pressure sensor 17A and the second pressure sensor 17B detect the pressures in the palm-side cuff 71, the sensing cuff 73, and the back-side cuff 74, and outputs, to the control unit 55, electrical signals corresponding to the pressures (step ST24). Based on the received electrical signals, the control unit 55 determines whether the pressures in the internal spaces of the palm-side cuff 71, the sensing cuff 73, and the back-side cuff 74 have reached a predetermined pressure for measurement of the blood pressure (step ST25). For example, in a case where the internal pressures of the palm-side cuff 71 and the back-side cuff 74 have not reached the predetermined pressure and the internal pressure of the sensing cuff 73 has reached the predetermined pressure, the control unit 55 closes the first on-off valve 16A and feeds compressed air through the first flow path 7a.

When the internal pressures of the palm-side cuff 71 and the back-side cuff 74 and the internal pressure of the sensing cuff 73 all have reached the predetermined pressure, the control unit 55 stops driving the pump 14 (YES in step ST25). At this time, as illustrated in FIGS. 18 and 19, the palm-side cuff 71 and the back-side cuff 74 are sufficiently inflated, and the inflated palm-side cuff 71 presses the back plate 72.

Additionally, the back-side cuff 74 presses against the curler 5 in a direction away from the wrist 200, and then the belt 4, the curler 5, and the device body 3 move in a direction away from the wrist 200, and as a result, the palm-side cuff 71, the back plate 72, and the sensing cuff 73 are pulled toward the wrist 200 side. In addition, when the belt 4, the curler 5, and the device body 3 move in a direction away from the wrist 200 due to the inflation of the back-side cuff 74, the belt 4 and the curler 5 move toward both lateral sides of the wrist 200, and the belt 4, the curler 5, and the device body 3 move in a state of close contact with both lateral sides of the wrist 200. Thus, the belt 4 and the curler 5, which are in close contact with the skin of the wrist 200, pull the skin on both lateral sides of the wrist 200 toward the hand back side.

Additionally, as the belt 4 and the curler 5 move toward both sides of the wrist 200, the prong 61e of the buckle 61c is pressed against the edge of the small hole 62a against which the prong 61e has abutted, as illustrated in FIG. 24. Thus, the prong 61e applies a load to the edge of the small hole 62a. The direction of action of the load is parallel to or substantially parallel to the direction orthogonal to the side surface 69c.

The sensing cuff 73 is inflated by being fed with a predetermined amount of air such that the internal pressure equals the pressure required to measure blood pressure, and is pressed toward the wrist 200 by the back plate 72 that is pressed by the palm-side cuff 71. Thus, the sensing cuff 73 presses the artery 210 in the wrist 200 and occludes the artery 210 as illustrated in FIG. 19.

Additionally, the control unit 55, for example, controls the second on-off valve 16B and repeats the opening and closing of the second on-off valve 16B, or adjusts the degree of opening of the second on-off valve 16B to pressurize the internal space of the palm-side cuff 71. In the process of pressurization, based on the electrical signal output by the second pressure sensor 17B, the control unit 55 obtains measurement results such as blood pressure values, for example, the systolic blood pressure and the diastolic blood pressure, and the heart rate and the like (step ST26). The control unit 55 outputs an image signal corresponding to the obtained measurement results to the display unit 12, and displays the measurement results on the display unit 12 (step ST27). In addition, after the end of the blood pressure measurement, the control unit 55 opens the first on-off valve 16A and the second on-off valve 16B.

The display unit 12 receives the image signal, and then displays the measurement results on the screen. The user views the display unit 12 to confirm the measurement results. After the measurement is complete, the user removes the prong 61e from the small hole 62a. removes the second belt 62 from the frame body 61d, and removes the wrist 200 from the curler 5, thus removing the blood pressure measurement device 1 from the wrist 200.

When the blood pressure measurement device 1 according to one embodiment configured as described above is attached to the wrist 200, the belt 4 curves in such a manner as to follow to the shape of the wrist 200. Furthermore, a part of the second portion 61g2 of the prong 61e abuts against a portion of the wrist 200-side edge of the small hole 62a which portion is located on the support portion 61f side in the longitudinal direction of the second belt 62. Inflation of the cuff structure 6 pulls the belt 4 in a direction in which the belt 4 is spread, and a load is input to the first belt body 63 from the prong 61e. Pulling the belt 4 in the spreading direction results in increase of the load applied to the first belt body 63 from the prong 61e. At this time, the load applied from the prong 61e acts toward the hole reinforcing portion 69.

However, in the present embodiment, the configuration is such that the first surface 69a of the hole reinforcing portion 69 is disposed on the wrist 200 side with respect to the second surface 69b. Thus, the side surface 69c faces the portion of the wrist 200-side edge of the small hole 62a which portion is located on the support portion 61f-side of the second belt 62 in the longitudinal direction of the second belt 62. As a result, the hole reinforcing portion 69 receives, on the side surface 69c. a part of the load applied from the prong 61e. Stress is received by the surface to enable suppression of stress concentration, thus allowing suppression of cracking in the first belt body 63. Consequently, durability of the second belt 62 can be improved.

Furthermore, a part of the load applied from the prong 61e also acts on a corner portion formed by the first surface 69a and the side surface 69c. but the corner portion is constituted to be obtuse, allowing suppression of stress concentration on the corner portion. Thus, possible cracking around the corner portion can be prevented. Consequently, durability of the second belt 62 can be improved.

In a case where the second insert 66 is formed by stamping using pressing, burrs may be formed at edges of the second surface 69b. which is opposite to the surface against which the mold is pressed. However, the second surface 69b is disposed opposite to the wrist 200 across the first surface 69a. thus allowing prevention of concentration, on the burrs, of the load from the prong 61e. The load from the prong 61e can be prevented from concentrating on the burrs, enabling prevention of possible cracking in the second belt 62. This allows the durability of the second belt 62 to be improved.

Furthermore, the thickness of the second belt 62 between the small hole 62a and the hole 68 along the longitudinal direction, in other words, the thickness of the second belt body 65 between the small hole 62a and the hole reinforcing portion 69 in the cross section illustrated in FIG. 24, is set being sufficient to reduce deformation caused by the load input from the prong 61e during the use of the blood pressure measurement device 1. This allows prevention of cracking of the outer surface of the second belt 62 caused by the deformation during repeated use of the blood pressure measurement device 1. Thus, the outer surface of the second belt 62 can be prevented from being cracked, allowing the durability of the second belt 62 to be improved.

Additionally, the belt 4 includes the second insert 66 constituted by the material having a high tensile strength, allowing elongation of the belt 4 to be suppressed even in a case where the cuff structure 6 is inflated to apply stress in the direction in which the belt 4 is pulled.

In addition, the first belt body 63 forming the outer surface of the belt 4 is constituted by the resin material, and thus in an operation of attaching the blood pressure measurement device 1, ease of the attachment can be ensured due to the flexibility of the resin material. Specifically, when the entire belt 4 is constituted by a material having a high tensile strength in order to make elongation difficult, the flexibility during attachment is degraded, but with employing a configuration in which the second insert 66 constituted by a material having a higher tensile strength than the first belt body 63 is provided in the first belt body 63 constituting the outer surface, which is made of the resin, both ease of attachment and difficulty of elongation can be provided in a compatible manner. Consequently, accurate blood pressure measurement can be achieved by suppressing the elongation of the belt 4 while maintaining close contact in a case where the cuff structure 6 is inflated during blood pressure measurement.

### Second Embodiment

Now, a second embodiment of the blood pressure measurement device 1 will be described using FIG. 25. Note that the blood pressure measurement device 1 according to the second embodiment is configured such that a pin 112 is inserted into the small hole 62a in the second belt 62 for connection of the first belt 61 and the second belt 62, and in this regard, differs from the configuration in which the prong 61e of the buckle 61c of the first belt 61 is inserted into the small hole 62a. Thus, components of the blood pressure measurement device 1 of the second embodiment that are similar to the corresponding components of the blood pressure measurement device 1 according to the first embodiment described above are denoted by the same reference signs in the description, and descriptions and illustrations of these components are omitted as appropriate.

FIG. 25 is a cross-sectional view illustrating the second belt 62 of the blood pressure measurement device 1 according to the second embodiment. As illustrated in FIG. 25, in the blood pressure measurement device 1 according to the second embodiment, a butterfly buckle 110, for example, is used in place of the buckle 61c for connection of the first belt 61 and the second belt 62. The butterfly buckle 110 includes a first fixing portion fixed to the first belt 61 and a second fixing portion 111 fixed to the second belt 62.

The second fixing portion 111 includes a pin 112 inserted into the small hole 62a. The second fixing portion 111 is fixed to the second belt 62 in a state in which the pin 112 is inserted into the small hole 62a. The second fixing portion 111 is disposed on a surface of the second belt 62 opposite to the wrist 200. A part of the pin 112 faces, in the longitudinal direction of the second belt 62, the portion of the wrist 200-side edge of the small hole 62a which portion is located on the support portion 61f side in the longitudinal direction of the second belt 62. Note that a tip of the pin 112 protrudes out of the small hole 62a. as an example.

In the blood pressure measurement device 1 according to the second embodiment configured as described above, in a state in which the belt 4 is attached to the wrist 200 curving in such a manner as to follow to the circumferential surface of the wrist 200 and the cuff structure 6 is inflated, a part of the pin 112 abuts against the portion of the wrist 200-side edge of the small hole 62a which portion is located on the support portion 61f-side in the longitudinal direction of the second belt 62 to apply a load.

Thus, as is the case with the first embodiment, the hole reinforcing portion 69 receives, on the side surface 69c. a part of the load applied from the pin 112. The load is thus received by the surface, allowing suppression of stress concentration in the second belt body 65. As a result, cracking in the second belt body 65 can be suppressed, and thus, durability of the second belt 62 can be improved.

### Third Embodiment

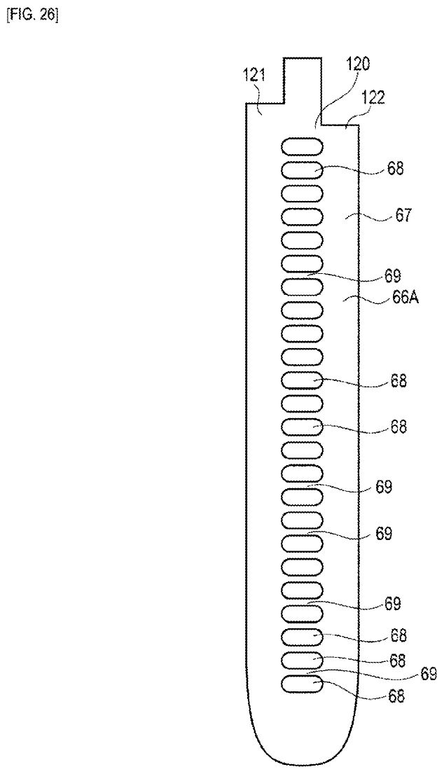

Now, a third embodiment of the blood pressure measurement device 1 will be described using FIG. 26. Note that the blood pressure measurement device 1 according to the third embodiment is configured such that the second insert 66 of the second belt 62 does not include the fitting portion 67a and is partially formed in a left-right asymmetric shape, and differs, in this regard, from the blood pressure measurement device 1 of the first embodiment including the fitting portion 67a. Thus, components of the blood pressure measurement device 1 of the third embodiment that are similar to the corresponding components of the blood pressure measurement device 1 according to the first embodiment described above are denoted by the same reference signs in the description, and descriptions and illustrations of these components are omitted as appropriate.

FIG. 26 is a plan view illustrating the configuration of the second insert 66. As illustrated in FIG. 26, the second insert 66 of the third embodiment is not provided with the fitting portion 67a. Furthermore, a part of the second insert 66 is constituted in a left-right asymmetric shape. The second insert 66 of the third embodiment has the same shape as that of the second insert 66 of the first embodiment except for the portion constituted in a left-right asymmetric shape.

In the second insert 66 of the third embodiment, for example, a shoulder portion 120 corresponding to portions on both sides in the width direction of the third hole portion 62b in the second insert 66 is constituted to be left-right asymmetric. As a specific example, the shoulder portion 120 includes a first shoulder portion 121 on one side in the width direction with respect to the third hole portion 62b and a second shoulder portion 122 on the other side in the width direction with respect to the third hole portion 62b. The second shoulder portion 122 is disposed, relative to the first shoulder portion 121, at a position biased toward one end opposite to the third hole portion 62b in the longitudinal direction of the second insert 66.

Thus, a mold used to form the second belt 62 of the blood pressure measurement device 1 according to the third embodiment includes a portion that cooperates with the shoulder portion 120 in positioning the second insert 66 within the mold such that the first surface 69a of the hole reinforcing portion 69 is disposed on the wrist 200 side with respect to the second face 69b.

As an example of this portion, a first mold 221 used to form the primary molded article 62A may be configured to be provided with a recess portion into which the second insert 66 fits. The recess portion includes a portion into which the first shoulder portion 121 fits and a portion into which the second shoulder portion 122 fits. Thus, when the second insert 66 is installed in the first mold 221 in a vertically inverted orientation with respect to the correct orientation, the first shoulder portion 121 faces the portion of the first mold 221 in which the second shoulder portion 122 fits, and the second shoulder portion 122 faces the portion of the first mold 221 in which the first shoulder portion 121 fits. Thus, the second insert 66 does not fit into the recess portion of the first mold 221. As a result, a worker or the like can be aware that the second insert 66 is not in the correct orientation. Note that the “correct orientation of the second insert 66” as used herein refers to an orientation in which the first surface 69a of the hole reinforcing portion 69 is disposed on the wrist 200 side with respect to the second surface 69b.

The blood pressure measurement device 1 according to the third embodiment thus enables positioning of a position of the second insert 66 within the mold, similarly to the first embodiment.

Note that in the present embodiment, the example has been described in which the shoulder portion 120 of the second insert 66 is constituted in a left-right asymmetric shape, but no such limitation is intended. Other than the shoulder portion 120, for example, the other end portion of the second insert 66 may be constituted in a left-right asymmetric shape.

Note that the present invention is not limited to the embodiments described above. For example, in the blood pressure measurement device 1, the timings when the first on-off valve 16A and the second on-off valve 16B are opened and closed during blood pressure measurement are not limited to the timings in the examples described above, and can be set as appropriate. Additionally, although the example has been described in which the blood pressure measurement device 1 performs blood pressure measurement by calculating the blood pressure with the pressure measured during the process of pressurizing the palm-side cuff 71, no such limitation is intended and the blood pressure may be calculated during the depressurization process or during both the pressurization process and the depressurization process.

In addition, in the example described above, the configuration has been described in which the air bag 81 is formed by each of the sheet members 86, but no such limitation is intended, and for example, the air bag 81 may further include any other configuration in order to manage deformation and inflation of the palm-side cuff 71, for example.

Additionally, in the examples described above, the configuration is described in which the back plate 72 includes the plurality of grooves 72a. but no such limitation is intended. For example, for management of the likelihood of deformation and the like, the number, the depth, and the like of the plurality of grooves 72a may be set as appropriate, and the back plate 72 may be configured to include a member that suppresses deformation.

Additionally, in the example described above, the hole reinforcing portion 69 is disposed in the center of the portion between the small holes 62a in the second belt body 65, but no such limitation is intended. For example, as illustrated in FIG. 27, the hole reinforcing portion 69 may be disposed at a position biased toward the end portion opposite to the third small hole 62b in the longitudinal direction of the second belt 62.

In other words, the hole reinforcing portion 69 may be disposed at a position, in the longitudinal direction of the second belt 62, biased toward a portion of the wrist 200-side edge of the small hole 62a against which the prong 61e or the pin 112 abuts, the portion being located on the support portion 61f side in the longitudinal direction of the second belt 62.

This configuration reduces the thickness portion between the small hole 62a and the hole 68 along the longitudinal direction of the second belt 62. As a result, a deformation margin, deformed by the load applied from the prong 61e or the pin 112, can be reduced, and thus cracking of the surface of the second belt 62 can be suppressed.

Additionally, as illustrated in FIG. 28, the thickness of the hole reinforcing portion 69 may be increased. “Increasing the thickness” as used herein refers to an increase in thickness to the extent that the feel of the belt 4 attached to the wrist is not impaired and that the operation of attaching the belt 4 is not obstructed.

An increased thickness of the hole reinforcing portion 69 enables an increase in the rigidity of the hole reinforcing portion 69, thus allowing suppression of deformation of the hole reinforcing portion 69 during use of the blood pressure measurement device 1. As a result, deformation of a portion of the second belt body 65 near the hole reinforcing portion 69 can be suppressed, and thus cracking caused by the deformation can be suppressed. Consequently, durability of the second belt 62 can be improved.

Additionally, in the example described above, the configuration in which the second insert 66 is formed by stamping using pressing on a plate-shaped member has been described as an example, but no such limitation is intended. As another example, a configuration may be provided in which the second insert 66 is formed by blanking a plate-like member by laser machining, as illustrated in FIG. 29.

The side surface 69c of the hole reinforcing portion 69 of the second insert 66 formed by blanking the plate-like member by laser machining is constituted to have an inclined shape because heat generated by laser irradiation gradually decreases in the thickness direction of the plate-like member. As a result, like the hole reinforcing portion 69 of the second insert 66 of the blood pressure measurement device 1 in the first embodiment, the hole reinforcing portion 69 of the second insert 66 formed by blanking by laser machining is constituted to have a trapezoidal cross section in which an angle between one of the side surfaces 69c and the first surface 69a is obtuse, an angle between the other side surface 69c and the first surface 69a is obtuse, an angle between one of the side surfaces 69c and the second surface 69b is acute, and an angle between the other side surface 69c and the second surface 69b is acute.

Furthermore, both edges 69a1 of the first surface 69a are melted by heat of laser machining and thus both edges 69a1 of the first surface 69a are constituted into raised protruding portions compared to other portions of the first surface 69a. In addition, both edges 69b1 of the second surface 69b are constituted into raised protruding portions compared to other portions of the second surface 69b.

The blood pressure measurement device 1 including the second insert 66 configured as described above can improve the durability of the belt 4, similarly to the blood pressure measurement device 1 of the first embodiment described above. Furthermore, since both edges 69a1 of the first surface 69a are constituted into the protruding portions, cracking can be prevented from occurring inside the second belt 62 near both edges 69a1, thus allowing the durability of the second belt 62 to be improved. Furthermore, by forming the second insert 66 by laser processing, possible burrs can be prevented from occurring, thus allowing prevention of possible cracking in the second belt body 65 caused by the burrs.

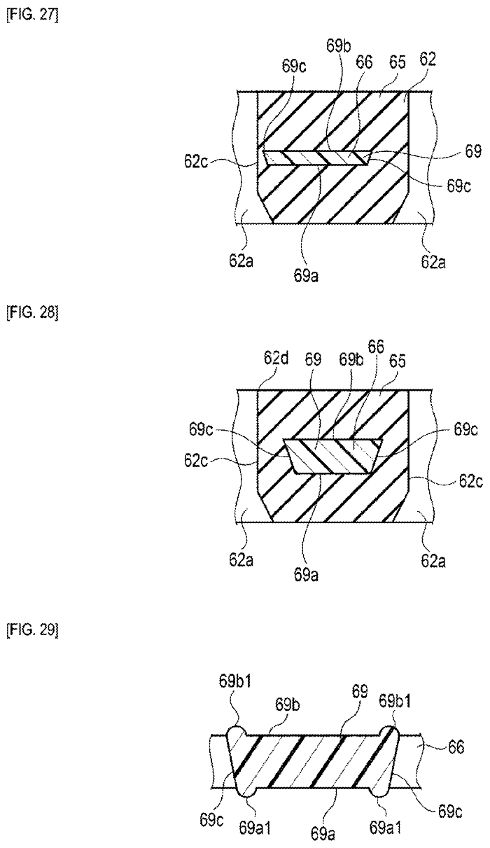

In the example described above, the cross-sectional shape of the hole reinforcing portion 69 of the second insert 66 is constituted like a trapezoid, but no such limitation is intended. As another example, as illustrated in FIG. 30, a cross section of the hole reinforcing portion 69 orthogonal to the width direction of the second belt 62 may be constituted like a rectangle, or an oblong figure as an example,

Additionally, the hole reinforcing portion 69 constituted to have an oblong cross section may be disposed such that the center of the cross section of the hole reinforcing portion 69 coincides with the center of a portion of a cross section of the second belt body 65 between the two adjacent small holes 62a which portion is orthogonal to the width direction of the second belt 62, as is the case with the first embodiment, as illustrated in FIG. 30.

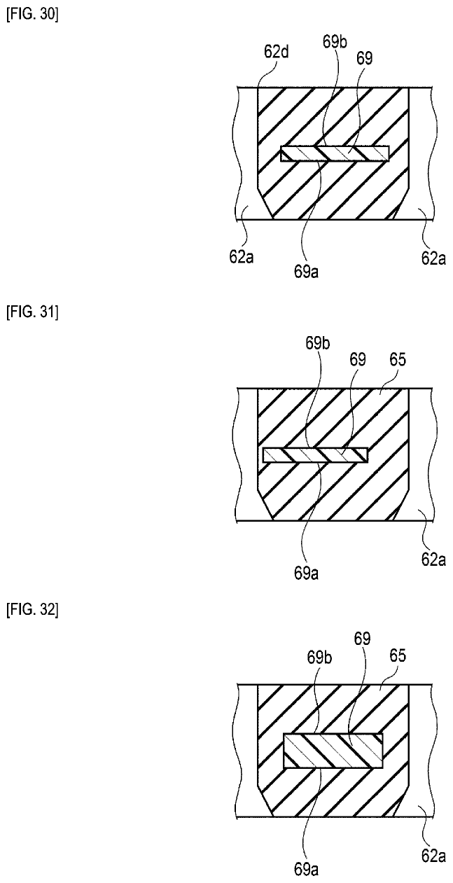

Alternatively, as illustrated in FIG. 31, the hole reinforcing portion 69 constituted to have an oblong cross section may be disposed at a position biased toward the small hole 62a. as is the case with a modified example illustrated in FIG. 27. Alternatively, as illustrated in FIG. 32, the thickness of the hole reinforcing portion 69 constituted to have an oblong cross section may be constituted to be large as is the case with a modified example illustrated in FIG. 28.

In other words, the embodiments described above are merely examples of the present invention in all respects. Of course, various modifications and variations can be made without departing from the scope of the present invention. Thus, specific configurations in accordance with an embodiment may be adopted as appropriate at the time of carrying out the present invention.

### REFERENCE SIGNS LIST

1 Blood pressure measurement device
3 Device body
4 Belt
5 Curler
6 Cuff structure
7 Fluid circuit
7a First flow path
7b Second flow path
7c Third flow path
11 Case
12 Display unit
13 Operation unit
14 Pump
15 Flow path unit
16 On-off valve
16A First on-off valve
16B Second on-off valve
17 Pressure sensor
17A First pressure sensor
17B Second pressure sensor
18 Power supply unit
19 Vibration motor
20 Control substrate
31 Outer case
31a Lug
31b Spring rod
32 Windshield
33 Base
35 Back lid
35a Screw
41 Button
42 Sensor
43 Touch panel
51 Substrate
52 Acceleration sensor
53 Communication unit
54 Storage unit
55 Control unit
61 First belt
61a First hole portion
61b Second hole portion
61c Buckle (connector)
61d Frame body
61e Prong
62 Second belt
62a Small hole (first hole)
62b Third hole portion
65 Second belt body (belt body)
66 Second insert (insert)
67a Fitting portion
68 Hole (second hole)
69 Hole reinforcing portion (positioning portion)
71 Palm-side cuff (cuff)
71B Pressing cuff
72 Back plate
72a Groove
73 Sensing cuff
74 Back-side cuff (cuff)
76 Bag-like cover body
81 Air bag (bag-like structure)
86 Sheet member
86a First sheet member
86b Second sheet member
86b1 Opening
86c Third sheet member
86c1 Opening
86d Fourth sheet member
91 Air bag (bag-like structure)
92 Tube
93 Connection unit
96 Sheet member
96a Fifth sheet member
96b Sixth sheet member
101 Air bag (bag-like structure)
102 Tube
103 Connection portion
106 Sheet member
106a Seventh sheet member
106b Eighth sheet member
106b1 Opening
106c Ninth sheet member
106c1 Opening
106d Tenth sheet member
106d1 Opening
106e Eleventh sheet member
106e1 Opening
106f Twelfth sheet member
106f1 Opening
106g Thirteenth sheet member
106g1 Opening
106h Fourteenth sheet member
106h1 Opening
106i Fifteenth sheet member
106i1 Opening
106j Sixteenth sheet member
106j1 Opening
106k Seventeenth sheet member
106k1 Opening
106l Eighteenth sheet member
110 Butterfly buckle (connector)
112 Pin
120 Shoulder portion (positioning portion)
200 Wrist
210 Artery
221 First mold
221a First pin

1. A belt comprising:
a first belt constituted in a band-like shape;
a second belt including a belt body and an insert, the belt body being constituted in a band-like shape using a resin material and including a plurality of first holes formed along a longitudinal direction, the insert being disposed in the belt body and including a plurality of second holes with a plurality of the first holes disposed on an inner side of the respective second holes, a cross section of a portion of the insert, which is between two adjacent second holes of the plurality of second holes and orthogonal to a width direction of the belt body, being constituted like a trapezoid with a short side being disposed on a living body side, the insert being formed of a material having a higher tensile strength than the resin material; and
a connector connecting the first belt and the second belt.

2. The belt according to claim 1, wherein the insert includes a positioning portion configured for positioning in a mold used to form the second belt.

3. A blood pressure measurement device comprising:
a belt including
a first belt constituted in a band-like shape,
a second belt including a belt body and an insert, the belt body being constituted in a band-like shape using a resin material and including a plurality of first holes formed along a longitudinal direction, the insert being disposed in the belt body and including a plurality of second holes with a plurality of the first holes disposed on an inner side of the respective second holes, a cross section of a portion of the insert, which is between two adjacent second holes of the plurality of second holes and orthogonal to a width direction of the belt body, being constituted like a trapezoid with a short side being disposed on a living body side, the insert being formed of a material having a higher tensile strength than the resin material, and
a connector connecting the first belt and the second belt; and
a cuff structure provided on an inner side of the belt and configured to be inflated with a fluid.
